SEQUENCE LISTING (4 sequences)

SEQ ID NO: 1 - DNA, 22 bp, Homo sapiens
  source 1..22: mol_type = other DNA
gaaatgccaccttttgacagtg

SEQ ID NO: 2 - DNA, 21 bp, Homo sapiens
  source 1..21: mol_type = other DNA
tggatgctctcatcaggacag

SEQ ID NO: 3 - DNA, 21 bp, Homo sapiens
  source 1..21: mol_type = other DNA
aggtcggtgtgaacggatttg

SEQ ID NO: 4 - DNA, 23 bp, Homo sapiens
  source 1..23: mol_type = other DNA
tgtagaccatgtagttgaggtca

(19) United States
(12) Patent Application Publication
(10) Pub. No.: US 2025/0229068 A1
(43) Pub. Date: Jul. 17, 2025

(54) ELECTROCONDUCTIVE MICRONEEDLE FOR DRUG DELIVERY INTO DEEP TISSUE

(71) Applicants: The University of Hong Kong, Hong Kong (HK); City University of Hong Kong, Hong Kong (HK)

(72) Inventors: Wei QIAO, Hong Kong (HK); Prasanna NEELAKANTAN, Chennai (IN); Sumanta GHOSH, Hong Kong (HK); Chenjie XU, Hong Kong (HK)

(21) Appl. No.: 19/019,510

(22) Filed: Jan. 14, 2025

Related U.S. Application Data
(60) Provisional application No. 63/621,121, filed on Jan. 16, 2024.

(51) Int. Cl. A61M 37/00

(52) U.S. Cl. CPC A61M 37/0015; A61M 2037/0023; A61M 2037/0046; A61M 2037/0053; A61M 2037/0061; A61M 2202/0007; A61M 2205/0205; A61M 2205/0233; A61M 2205/0244; A61M 2207/10; A61M 2210/04

(57) ABSTRACT

The present invention relates to a novel microneedle platform for minimally invasive delivery of therapeutic agents and the modulation of the neuro-immune axis in deep tissue. The present invention also adopts an innovative approach for minimally invasive and precisely controllable treatment of deep cutaneous diseases, conditions, and disorders using said electroconductive MN patch, which has the potential to benefit millions of patients worldwide.

### FIELD OF THE INVENTION

The present invention relates to an electroconductive microneedle for minimally invasive drug delivery into subcutaneous tissue, aimed at eradicating infections and modulating neuro-immune responses. This approach avoids invasive injections or surgeries, reducing the risks of secondary injury, discomfort, and complications.

### REFERENCE TO SEQUENCE DISCLOSURE

The sequence listing file under the file name “P3049US01_Sequence_listing.xml” submitted in ST.26 XML file format with a file size of 4.43 KB created on 9 Jan. 2025 and filed on 14 Jan. 2025 is incorporated herein by reference.

### BACKGROUND OF THE INVENTION

Skin infections have become one of the most devastating health issues, affecting 20-25% of the global population. Conventional therapeutic approaches face limitations in drug penetration through the skin barrier, resulting in insufficient local bioavailability in deep tissues and hindering effective treatment of deep tissue infections. High doses of potent drugs taken orally or systemically can cause severe adverse effects, including teratogenicity, hepatotoxicity, and systemic toxicity. Despite the severity of deep invasive infections, most research has focused on superficial infections, with limited strategies for managing deep infections.

Microneedle (MN) technology is widely used in drug delivery due to its minimally invasive nature, ease of access, reduced dosing frequency, and high tolerability. For instance, conventional antibiotics (such as, gentamycin, bleomycin) loaded into the MNs exhibits a constant plasma concentration for a longer time than that of intravenous/intramuscular injection, which reduces the effective therapeutic concentration and dosage frequency. However, conventional biodegradable polymer-based MNs have limitations, including a lack of responsiveness to internal or external stimuli, which affects the control of drug delivery. Furthermore, it remains technically challenging for MNs to effectively deliver therapeutic agents into deep tissue.

Recently externally controlled delivery strategies such as light/Near Infra-Red (NIR)-radiation, magnetic and electrical stimuli, have gained immense attention for their better control and precision-based on-demand release capability^{1,2}. However, several existing solutions fail to address key limitations. For instance, US20170028184A1³ discloses a microneedle platform with inherent anode and cathode properties for the cosmetic application. However, the complex design of the device composed of an electrically insulative substrate and the one electrically conductive layer comprises of an electrically conductive material carried on an outer surface of the electrically insulative substrate. In addition, the therapeutic efficiency of the MNs patch not explored in the same invention.

US20220193403A1⁴ discloses a wound dressing platform composed of one electrode in electrical communication with the therapeutic agent. The system also includes a power source in electrical communication with the one electrode and a vacuum source in fluid communication with the wound dressing. Lastly, the safety and efficacy issue of the devices was not evaluated.

WO2020060495A1⁵ discloses a conductive microneedle composed of acrylate-functionalized and other functionalized hydrophilic polymers, but the material compositions, such as methacrylate-functionalized hyaluronic acid or polyethylene glycol, differ fundamentally from the Gelatin Methacryloyl (GelMA) and carbon nanotube (CNT) combination in the present invention. Notably, WO2020060495A1 focuses on superficial drug delivery, particularly local anesthetic agents, without addressing deeper tissue drug delivery strategies or applications like fungal infections. Additionally, the reported conductive polymers do not include carbon nanotubes, which substantially influence the conductivity, impedance, and drug delivery performance.

US20210386985A1⁶ describes biodegradable microneedles made of GelMA for sustained doxorubicin release. However, the microneedles lack any conductive additives and rely on passive drug diffusion through solubilization of the GelMA matrix, resulting in sustained release over several days. In contrast, the present invention incorporates carbon nanotubes to achieve iontophoresis-based rapid drug release under an applied electrical field. This on-demand and tunable delivery mechanism enables precise drug administration into deeper tissue layers, a feature absent in US20210386985A1. Additionally, the therapeutic scope of US20210386985A1 is limited to sustained drug delivery, with no emphasis on immune stimulation or deep tissue targeting.

U.S. Pat. No. 9,737,247B2⁷ discloses a microneedle array primarily designed for biosensing and drug delivery, featuring hollow needles with interior probe structures and electrical wires for stimulation. However, the therapeutic application is limited to analyte detection and superficial drug release, with no evidence of its efficacy for deep tissue drug delivery.

Therefore, there is a need for an innovative solution that overcomes the limitations of conventional microneedle technology in delivering therapeutic agents effectively into deep tissue.

### SUMMARY OF THE INVENTION

Accordingly, the present invention aims to develop an on-demand, cost-effective MN patch for minimally invasive delivery of therapeutic agents to the deep cutaneous sites, with the potential to modulate the neuro-immune crosstalk within this area.

The combination of MN technology with the direct electrical stimulation creates an electrical field-based electro-osmotic movement which enable the robust release of therapeutic molecule drugs (e.g., small molecules like miconazole nitrate, bacitracin, and mupirocin, as well as antimicrobial peptides, enzymes, and extracellular vesicles) into deep tissue layers. The amount of drugs released from the electroconductive MN patches can be tuned by modulating the applied voltage or the duration of application. As the current density increases, both the amount and rate of drug release enhance. Similarly, the release is also proportionally increased with the duration of the application. It should be noted, however, that tuning the applied current must be done within a specific range to avoid eliciting any discomfort or pain to the subject.

Additionally, by disrupting the negatively charged microbial cell walls and polysaccharides matrix components of the microbial biofilm, the electroconductive MN patches also improve the drug molecule penetration inside the dense biofilm niche. Furthermore, the electroconductive MNs can also stimulate the nociceptive neurons to modulate immune cells within the microenvironment for the clearance of microbial and the development of protective cutaneous immunity.

In one aspect, the present invention provides a polymer-based electroconductive microneedle patch for delivering a drug to a deep subcutaneous tissue, which includes a biocompatible substrate as a supporting structure, a microneedle array and at least one therapeutic agent loaded onto the multiple polymer-based electroconductive microneedles. The microneedle array includes a plurality of polymer-based electroconductive microneedles affixed to the substrate. The polymer contains a photocrosslinkable biocompatible material reinforced with one or more electroconductive materials. The polymer and the photocrosslinkable biocompatible material have a ratio between 100:5 to 100:10.

The microneedle array is configured to release the at least one therapeutic agent upon on-demand stimulation by an electrical current of 0.5 mA, so that the at least one therapeutic agent is able to be delivered to sites of deep cutaneous layers without causing any damage to surrounding tissues.

In one embodiment, the photocrosslinkable biocompatible material includes gelatin methacrylate (GelMA), hyaluronic acid methacrylate polymer, alginate methacrylate, or acrylates.

In one embodiment, the one or more electroconductive materials include carbon nanotube, graphene, MXenes, graphene oxides, or reduced graphene oxides.

In one embodiment, the one or more electroconductive materials provide enhanced electrical conductivity and mechanical reinforcement, achieving sufficient mechanical strength to penetrate the epidermis layer without deformation under a force of 0.5 N per needle.

In one embodiment, the biocompatible substrate comprises biodegradable polymers. Examples of the biodegradable polymers may include gelatin, alginates, collagen, dextran, chitosan, albumin, starch, cellulose, methyl cellulose, carboxymethyl cellulose, or hyaluronic acid.

In one embodiment, the at least one therapeutic agent includes antibiotics, peptides, nanoparticles, enzymes, extracellular vesicles, antifungal drugs, cationic therapeutic molecules, or a combination thereof.

Preferably, the at least one therapeutic agent includes miconazole nitrate, with a loading amount of approximately 400 μg per patch.

In one embodiment, the plurality of polymer-based electroconductive microneedles has a height of 500-800 μm, a tip radius of 1-10 μm, and a base width of 200-400 μm.

In one embodiment, the polymer-based electroconductive microneedle patch exhibits antimicrobial properties without causing local inflammation and accelerates wound healing by promoting collagen synthesis in fibroblasts.

In one embodiment, the collagen synthesis in the fibroblasts is further enhanced by a sensory neuron-derived calcitonin gene-related peptide.

In one embodiment, the polymer-based electroconductive microneedle patch releases at least 55% of the at least one therapeutic agent within 5 minutes under an applied current of 0.5 mA.

In another aspect, the present invention provides a method for manufacturing a polymer-based electroconductive microneedle patch, including synthesizing a gelatin methacrylate solution by reacting gelatin with methacrylic anhydride; preparing a polymer matrix by dispersing carbon nanotubes into the gelatin methacrylate solution at a carbon nanotube concentration of 5.0 mg/mL to 10.0 mg/mL; pouring the polymer matrix into a micromold; photo-crosslinking the polymer matrix under UV light to form solidified microneedles; and loading the solidified microneedles with at least one therapeutic agent.

In one embodiment, the at least one therapeutic agent includes antibiotics, peptides, nanoparticles, enzymes, extracellular vesicles, antifungal drugs, cationic therapeutic molecules, or a combination thereof.

In another embodiment, the method further including attaching the polymer-based electroconductive microneedle patch to a silver adhesive tape and connecting it to an external power source for electrical stimulation.

In another aspect, the present invention provides a method for treating deep subcutaneous diseases, conditions, and disorders. The method includes administering the polymer-based electroconductive microneedle patch to a subject; generating an electrical field by supplying a current density of 0.5 mA; and releasing at least one therapeutic agent into a deep subcutaneous tissue in response to the electrical field. The polymer-based electroconductive microneedle patch is configured to release the at least one therapeutic agent upon on-demand stimulation, so that the at least one therapeutic agent is able to be delivered to sites of deep cutaneous layers without causing any damage to surrounding tissues. The current applied to the polymer-based electroconductive microneedle patch contributes to transient loosening of tight junctions between the subject's skin cells, facilitating permeation of the at least one therapeutic agent into deeper skin layers.

In one embodiment, the deep subcutaneous diseases, conditions, and disorders include microbial infection, acne, cancer, diabetes, mycetoma, chromoblastomycosis, and keratitis.

Preferably, the deep subcutaneous diseases, conditions, and disorders is microbial infection. The polymer-based electroconductive microneedle patch is configured to disrupt negatively charged microbial cell walls and polysaccharides matrix components of a biofilm, improving penetration of the at least one therapeutic agent into dense biofilm niches and eradicating the microbial infection.

In one embodiment, the polymer-based electroconductive microneedle patch provides an antimicrobial efficacy against fungal infections caused by Candida albicans, achieving a fungal clearance rate of at least 99.9%.

In one embodiment, the polymer-based electroconductive microneedle patch demonstrates biocompatibility with the subject's skin keratinocytes and sustain cell viability greater than 90% after administration.

In one embodiment, the electrical field activates nociceptive sensory neurons in the deep subcutaneous tissue, promoting immune responses via a neuroimmune axis.

In yet another aspect, the present invention provides a method for promoting tissue healing and activating subcutaneous protective immunity through modulation of the neuroimmune axis. The method includes applying the above polymer-based electroconductive microneedle patch to a subject. The polymer-based electroconductive microneedle patch is driven by an electric field.

The new approach presents several advantages, including but not limited to:
(1) The release of the drug is specifically regulated by an externally applied electric field, allowing for precise dosage control;
(2) Unlike traditional methods, this approach successfully achieves drug delivery to the deep subcutaneous layer of the skin;
(3) Unlike injection or surgical procedures commonly used in managing deep tissue infections, this approach minimizes the risk of secondary injury and discomfort; and
(4) By enhancing drug delivery and targeting the affected area, the treatment promotes higher drug utilization rates and reduces the overall duration of therapy. This leads to minimized side effects associated with antifungal treatments.

### BRIEF DESCRIPTION OF THE DRAWINGS

Embodiments of the invention are described in more details hereinafter with reference to the drawings, in which:

FIG. 1A shows a schematic diagram showing the procedure for the synthesis of CNT reinforced GelMA and fabrication schemes of antifungal agent (e.g., miconazole nitrate) loaded MNs consists of GelMA and CNT. FIG. 1B shows the micro-needle structure, composition, and the operating mechanism of the present invention;

FIG. 2 shows ¹H NMR of GelMA and Gelatin showing the successful conjugation of methacrylate groups on the gelatin backbone;

FIG. 3A shows dimensions and optical images of the electroconductive MN patch (scale bar=10 mm). FIG. 3B shows scanning electron microscopy (SEM) images of the MNs showing successful fabrication of pyramidal-shaped MNs. Scale bar=1 mm;

FIG. 4A shows fourier transform infrared (FTIR) spectrum of GelMA and GelMA+CNT MNs. FIG. 4B shows mechanical properties of the MNs with respect to per needle, (n=3);

FIG. 5 shows platform schematics of deep cutaneous infection development by Candida albicans and its treatment strategy using miconazole nitrate loaded electroconductive MNs (M-MNs). E, D and S represent epidermal, dermal, and subcutaneous layers of the skin tissue, respectively;

FIG. 6A shows a schematic illustration of the testing platform for the electrical conductivity measurement. FIG. 6B shows electrical impendence of the different GelMA+CNT MNs compared to pure GelMA (n=6 per group). FIG. 6C shows electrical conductivity of the different GelMA+CNT MNs compared to pure GelMA (n=6 per group). FIG. 6D shows constant current flow in the different GelMA+CNT electroconductive MNs over 5 min timespan;

FIG. 7A shows Cy-3 release performance from the electroconductive MNs (n=6 each group). FIG. 7B shows hemolysis assay of the fabricated MNs exhibiting blood compatibility (n=3 each group);

FIG. 8 shows ex vivo porcine skin penetration of the electroconductive MNs. The upper and lower panels represent brightfield and fluorescent images, respectively. Scale bar=500 μm;

FIG. 9 shows a schematic of drug release study procedure, and a HPLC standard curve of miconazole nitrate;

FIG. 10 shows cumulative % drug release from the electroconductive microneedles with and without the current supply (n=3 per group);

FIG. 11A shows colony forming unit (CFU) assay of M-MNs with current against planktonic Candida albicans (SC 5314) fungal cells. FIG. 11B shows representative optical images of CFU plates showing antifungal activity of corresponding groups. FIG. 11C shows confocal laser scanning microscopy (CLSM) images of live/dead staining images showing the potent fungicidal activity of M-MN+current compared to other groups, (scale bar, 20 μm). FIG. 11D shows CFU assay of the electroconductive M-MNs against planktonic Candida albicans (SC 5314) fungal cells;

FIG. 12A shows a schematic illustration of antibiofilm activity testing platforms. FIG. 12B shows CFU assay of M-MNs with current against 3 days old Candida albicans (SC 5314) biofilm. FIG. 12C shows representative optical images of CFU plates showing complete eradication of C. albicans biofilm after M-MNs with current application compared to other groups;

FIG. 13 shows CLSM images of the Cy-3 (model drug) penetration from the M-MNs+Current in an in-vitro biofilm model (consisting of GFP-C. albicans) after 0.5 mA current supply for 5 min;

FIGS. 14A-14C show in vitro drug release variation into the C. albicans biofilm with respect to current density according to an embodiment of the present invention. FIG. 14A shows an illustration of the in vitro model. FIG. 14B shows representative 2D, and cross-sectional (insets) CLSM images of Cy-3 (model drug molecule) release variation into the 3-days old C. albicans biofilm from the electroconductive GelMA+5.0 CNT MNs with respect to various current densities, compared to pure GelMA MNs. FIG. 14C shows statistical analysis of mean fluorescence intensity of Cy-3 released from GelMA+5.0 CNT MNs inside the biofilm with different current stimulation;

FIG. 15A shows in vitro deep tissue infection model for testing the antifungal efficiency of the fabricated microneedles. Agarose gel (1.4 wt. %) mimics the natural dermal barrier above the fungal biofilm. FIG. 15B shows CLSM images of deep antifungal activity of M-MNs with current compared to other groups. FIG. 15C shows quantitative analysis of fluorescence intensity of the CLSM images;

FIG. 16A shows a schematic of the ex-vivo study plan. FIG. 16B shows representative optical images of CFU plates of the ex-vivo study. FIG. 16C shows CFU counting of ex-vivo antimicrobial activity of MNs on an ex-vivo porcine skin model;

FIG. 17 shows in vitro biocompatibility of electroconductive MNs on skin keratinocyte (HaCaT) cells according to an embodiment of the present invention;

FIG. 18A shows an illustration of animal study design, insets images are the photographs of nodule formation fungi infection development and M-MNs application process. FIG. 18B shows a statistical comparison of nodule area reduction after different treatments with respect to 5 days timespan (n=6 per group). FIG. 18C shows representative optical images of the nodules after different treatments (scale bar=5 mm). FIG. 18D shows optical images of CFU plates of different organ specimens after day 6 with different treatment groups (n=6). FIG. 18E shows an illustration of nodule area reduction after day 6 with different treatment groups. FIG. 18F shows fungal burden (CFU counting) of the mice skin after day −6 (n=3 per group). FIG. 18G shows fungal burden (CFU counting) of the mice blood after day 6. (n=3 per group);

FIG. 19A shows fungal burden (CFU counting) of the mice kidney after day 6 (n=3 per group). FIG. 19B shows quantitative analysis of infiltrated inflammatory cells in different treatment groups after HE staining;

FIG. 20 shows visual confirmation of pus-like debris formation beneath the skin after the respective application on day 6 according to an embodiment of the present invention;

FIG. 21 shows representative hematoxylin and eosin (HE) staining images of the mice skin tissue sections after day 6 with different treatments;

FIG. 22 shows representative HE staining images of the different organ tissue sections after day 6 with different treatments, depicting the biosafety of different treatment groups (n=3 per group, scale bar=50 μm);

FIG. 23 shows representative periodic acid staining (PAS) images of the inoculated mice skin tissue sections with different treatments. Scale bar=500 μm, 25 μm;

FIG. 24 shows representative Masson's trichrome staining images of mice skin tissue sections after different treatments. E, D and S represent epidermal, dermal, and subcutaneous layers of the skin tissue, respectively;

FIG. 25 shows expression of CGRP gene after different treatments (n=3 per group) on PC-12 cells after 24 h by RT-PCR according to an embodiment of the present invention; and

FIG. 26A shows representative immunohistochemical (IHC) staining images of the mice skin tissue sections after day −6 with different treatments. FIG. 26B shows representative IHC staining images of the mice skin tissue sections after day −6 with different treatments. FIG. 26C shows representative IHC staining images of the mice skin tissue sections after day 6 with different treatments. FIGS. 26D-26E show quantitative analysis of the number of IL-23 positive DC cells, and IL-17A positive immune cells after different treatments (n=3 per group). FIG. 26F shows a schematic illustration of neuro-immune interaction driven by the electroconductive MNs explored in the present invention.

### DETAILED DESCRIPTION

In the following description, electroconductive MN patches are set forth for the deep cutaneous infection as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The application of newer generation effective transdermal drug delivery systems such as microgel depot, microneedles are unsuccessful due to the subtherapeutic level drug delivery, poor biocompatibility, and uncontrolled targeted release. Several strategies have been explored to overcome the challenges of deep tissue delivery. However, previous studies on microneedle-mediated deep tissue delivery primarily focus on modifying microneedle length, increasing drug dosage, or incorporating targeted nanoparticles. Each of these methods has inherent limitations and may result in additional adverse effects.

To address these challenges, the present invention has developed an electroconductive MNs system composed of biocompatible GelMA and CNT and ensure its painless deep tissue puncturing ability owing to its phenomenal mechanical characteristics.

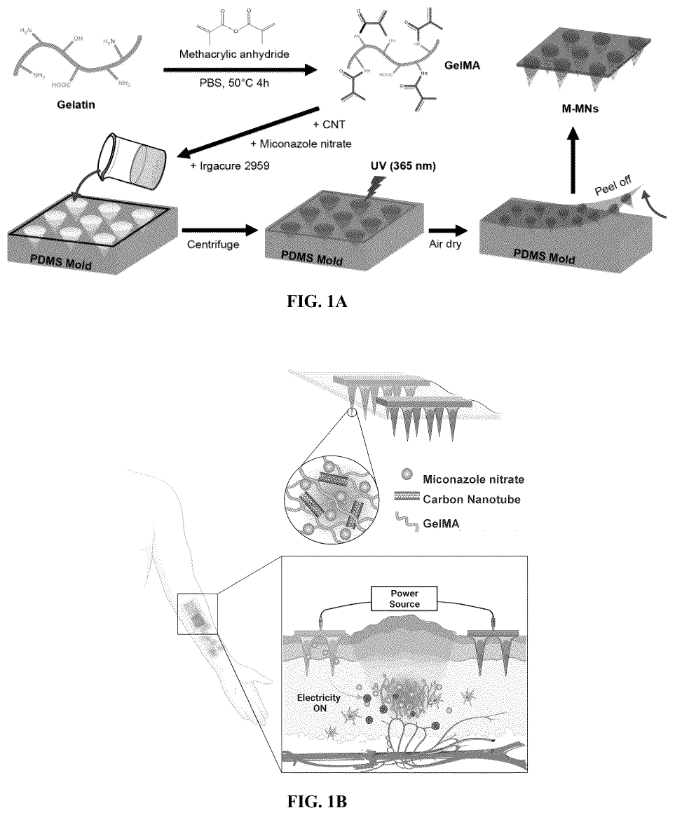

In this line of investigation, a biocompatible electrically stimulated, minimally invasive MN platform has been developed, which can efficiently deliver drug molecules to deep subcutaneous tissue layers and efficiently eradicate the deep infection. The application of low-voltage current stimulation to improve deep tissue penetration represents a novel and non-obvious approach (FIG. 1B).

The present invention offers a novel approach to chemotherapy by utilizing an electroconductive microneedle patch for direct electrical field-based drug delivery in humans. This polymer-based electroconductive microneedle patch exhibits excellent mechanical strength, drug-loading capacity, and electroconductive properties. When activated by an external electrical field, the patch enables precise delivery of therapeutic agents to deep subcutaneous tissues without causing side effects. Key innovations include: (1) electroconductivity, allowing the microneedle patch to channel the applied electrical field into the skin; (2) targeted drug delivery, where the electric field drives precise release of drugs to deeper tissues without damaging surrounding areas; and (3) enhanced therapeutic effects, especially for deep fungal infections that are resistant to conventional treatments.

More specifically, the polymer-based microneedle patch of the present invention consists of a substrate as a supporting structure, multiple polymer-based electroconductive microneedles affixed to the substrate, and at least one therapeutic agent(s) loaded onto the multiple polymer-based electroconductive microneedles.

The substrate is made from biodegradable polymers supporting a microneedle array. Examples of the biodegradable polymers include gelatin, alginates, collagen, dextran, chitosan, albumin, starch, cellulose, methyl cellulose, carboxymethyl cellulose, or hyaluronic acid.

The polymer includes, but is not limited to, any photocrosslinkable biocompatible materials. Examples of the photocrosslinkable biocompatible materials may be GelMA, hyaluronic acid methacrylate polymer reinforced with highly electroconductive materials such as CNT. The CNT is at an optimized concentration of 5.0 mg/mL-10.0 mg/mL to enhance mechanical and electrical properties. For instance, the concentration of CNT can be 5.5 mg/mL, 6.0 mg/mL, 6.5 mg/mL, 7.0 mg/mL, 7.5 mg/mL, 8.0 mg/mL, 8.5 mg/mL, 9.0 mg/mL, or 9.5 mg/mL.

In one embodiment, the microneedles can be designed in different shapes as needed, such as a pyramid shape. The microneedles exhibit dimensions suitable for deep penetration, with a height of 500-800 μm, a tip radius of 1-10 μm, and a base width of 200-400 μm.

In one embodiment, the microneedles possess sufficient mechanical properties to penetrate the epidermis layer of skin without causing pain. When an external electric field is applied to the microneedle patch, it stimulates iontophoresis and electroporation, enhancing the permeability of the skin. Meanwhile, the therapeutic agents loaded onto the microneedles are released and effectively reach the deeper subcutaneous layer of the skin driven by the electric field.

The electrical stimulation induces iontophoresis and electroporation mechanisms, which increase the permeability of skin barriers and facilitate deeper tissue penetration. For instance, the electroconductive MN patch of the present invention exhibits excellent electrochemical performance allowing on-demand delivery of a model drug, Cyanine-3 (Cy3), into deep tissue layers of porcine skin.

Also, the applied electric stimulation improves the influx of cationic antifungal molecules (such as, miconazole nitrate, antimicrobial peptides, nanoparticles, nanorobots, exosomes, enzymes, etc) inside the negatively charged extracellular polymeric substances (EPS) of biofilms through the means of electro-osmotic flow. The antifungal efficacy of the electroconductive MNs is tested using in vitro and ex vivo models that mimic deep fungal infection.

The in vitro studies demonstrate that M-MNs show superior biofilm penetration capability and fungicidal efficacy compared to miconazole cream or M-MNs without electric stimulation. The ex vivo tests of the electroconductive M-MNs show their increased effectiveness in eradicating C. albicans infections in deep tissue layer of porcine skin compared to cream or M-MNs alone. Moreover, the anti-microbial and pro-healing effects are confirmed in a murine subcutaneous fungal infection model. The in vivo studies using a mouse model validate that the application of electrically-stimulated M-MNs facilitates the delivery of one or more therapeutic agents to deeper tissue level to eliminate microbial infection.

The microneedle patch, propelled by an electric field, enables an on-demand delivery of one or more therapeutic agents into deep subcutaneous tissues to eliminate deep infection.

The one or more therapeutic agents include, but is not limited to, antibiotics, peptides, nanoparticles, enzymes, extracellular vesicles, and other substances capable of treating deep cutaneous diseases, conditions, and disorders.

In one embodiment, the deep cutaneous diseases, conditions, and disorders include, but are not limited to, microbial infections, such as those caused by bacteria, viruses, fungi, or parasites, capable of causing diseases. In particular, the electroconductive MNs of the present invention are employed to combat the microbial infections but the application is not limited to any subcutaneous disease such as acne, cancer, diabetes, mycetoma, chromoblastomycosis, and keratitis.

Moreover, the patch demonstrates the capacity to foster tissue healing and activating subcutaneous protective immunity through modulation of the neuroimmune axis. Additionally, the electric field-driven microneedle patch can also prevent secondary injury or uncomfortable resulting from injection and surgical procedures. Therefore, the polymer-based electroconductive microneedle patch exhibits the capability to effectively manage diseases in clinical practice.

In another embodiment, the results also show that combining an antimicrobial agent, such as miconazole, with direct current application synergistically stimulated nociceptive sensory nerves, activating protective cutaneous immunity mediated by dermal dendritic and T6-T cells. The synergistic action of antifungal drugs with the electroconductive MNs can completely eliminate the deep cutaneous fungal infection in the in vivo mice model. In addition, in vivo application of electroconductive MN patches also ensure the prevention of spreading of the infection into the systemic circulation or other organs.

Furthermore, the present invention also offers a novel therapeutic approach that distinguishes itself from conventional methods. The manufacturing process of this approach is inexpensive, and streamline, thereby the resulting product is convenient to store. As a result, it alleviates the financial burden on patients and the healthcare system.

### EXAMPLE

### Example 1

### Material and Methods

### Real-Time Quantitative Polymerase Chain Reaction (RT-qPCR)

PC-12 cells are cultures with 10% FBS and 1% Penicillin-Streptomycin supplemented with DMEM and incubated in a humidified incubator (37° C., 5% CO₂). For the experiment, 5×10⁴ cells per well are seeded on to 24 well plates and incubate for 24 hours to form a uniform monolayer. After that, miconazole nitrate (400 g/mL) and electrical stimulation (0.5 mA, 5 mins) are given as treatment. Further, untreated cells are served as a control. After the treatment, medium is changed for electrical stimulation group and incubate for another 24 h. After that, total RNA is extracted from the treated cells using RNeasy Mini Kit (Qiagen, Hilden, Germany) and checked the purity using NanoDrop (Thermo Fisher Scientific, USA). Next, RNA is reversely transcribed into cDNA using the First Strand cDNA kit (Takara, DaLian, China). Real-time quantitative PCR reaction is performed with cDNA using TB green Premix. The mean cycle threshold (Ct) value of each target gene is normalized to the housekeeping genes (i.e., GAPDH).

The primers sequences for CGRP and GAPDH used are as follows:

| Genes | Primer sequences |
|---|---|
| CGRP | Forward: 5′-GAAATGCCACCTTTTGACAGTG-3′ |
| | (SEQ ID NO.: 1) |
| | Reverse: 5′-TGGATGCTCTCATCAGGACAG-3′ |
| | (SEQ ID NO.: 2) |
| GAPDH | Forward: 5′-AGGTCGGTGTGAACGGATTTG-3′ |
| | (SEQ ID NO.: 3) |
| | Reverse: 5′-TGTAGACCATGTAGTTGAGGTCA-3′ |
| | (SEQ ID NO.: 4) |

### Histology and Immunofluorescence Analysis

Immunostaining is performed using a standard protocol. Briefly, the skin specimens are incubated with primary antibodies to Anti-CD301b MGL2 rat monoclonal (Abcam, Cambridge, UK, ab14-3011-82, 1:50), Anti IL-23 rabbit polyclonal (Abcam, ab45420, 1:100), Anti-CGRP (Abcam, ab36001, 1:100), and Anti-IL-17A (Abcam, ab79056, 1:250) overnight at 4° C. Alexa-Fluor 488-conjugated and Alexa-Fluor 647-conjugated secondary antibodies (Thermo Fisher Scientific, Waltham, MA, USA) are used for immunofluorescent staining, while the nuclei are counterstained with Hoechst 33324 (Thermo Fisher Scientific). Immunofluorescent images are captured using an LSM 780 confocal microscope (Zeiss, Oberkochen, Germany).

### Biofilm Eradication Efficacy of the Electroconductive MNs

An in-house developed in-vitro fungal biofilm model is used. Briefly, three-day-old biofilms are prepared by culturing 1×10⁶ cells of GFP-candida in chamber slides. Subsequently, to mimic a deep skin fungal infection model, an agarose gel (1.4% weight, 1.5 mm thickness) is placed over the biofilm, and current is supplied through the miconazole nitrate-loaded MNs using the method described earlier. After that, the biofilms are incubated for another 4 hours at 37° C. to observe the drug-killing effect. The antimicrobial efficacy of the electroconductive MNs is assessed by examining the biofilm matrix under a confocal laser scanning microscope. Z-stack images are obtained from 5 different fields. Interestingly, the antimicrobial efficiency of the conductive MNs is also compared with that of the commercially available 2% miconazole nitrate gel (Zarin™) to gain insight into drug penetration and microbial eradication.

### Mechanical Characterizations of Electroconductive MNs

The mechanical strength of the patch is examined through a compression test using an Instron 5543 Tensile Meter. Briefly, the compressive force is applied perpendicularly to the MN tips which are placed upward direction through a flat-head stainless steel cylindrical probe with 5 mm diameter, at a constant speed of 0.5 mm·min¹. The displacement is measured until a pre-set maximum force of 50N per MN patch is achieved.

### Electrical Properties Evaluation of Electroconductive MNs

The electrical conductivity of the GelMA+CNT MNs is evaluated by measuring the electrical impendence of the MNs using a handheld multimeter (Escort EDM-169) and by placing the MNs on the 1.4 wt % agarose gel as an in-vitro replica model of human skin. The backside of the MNs is attached to a conductive silver tape for connecting to the positive electrode. The negative electrode of the multimeter is directly attached to the agarose gel substrate to ensure electric flow. A constant distance of 4 mm is maintained between the two electrodes throughout the experimental procedure to ensure the constant current flow in all samples. Further, the electrical conductivity of the pyramidal shapes MNs array is calculated using the following formula:

$$Electrical conductivity ( σ ) = 1 / ( R \times A / L ) ,$$

where R is the electrical impedance, A is the cross-sectional area of the MNs, and L is the length of the MNs.

Notably, for the square based pyramidal MNs, the area is calculated as A=s², where s is the length of one side of the MN base.

### Hemolysis Assay

The blood compatibility of the fabricated MNs is verified through a hemolysis assay conducted according to the ISO 10993-4:2017 standard. Briefly, Red blood cells (RBC) are isolated from the fresh, heparinized mice blood by centrifugation at 13000 rpm at 4° C., after which the supernatant is carefully removed, and the remaining RBC suspension is diluted with phosphate-buffered saline (PBS) to get the 20× diluted RBC suspension. Then, each MN patch is immersed with 2 mL of RBC suspension. For comparison, 1.6 mL of PBS with 0.4 mL of RBC suspension is taken as the negative control, while 1.6 mL of DI water with 0.4 mL of RBC suspension is taken as the positive control for this experiment. All the tubes are allowed to incubate for 2 hours at 37° C. and after those tubes are again centrifuged at 2500 rpm for 5 mins. Finally, after the centrifugation, absorbance is taken of supernatant for all tubes at 541 nm using a UV-visible spectrophotometer.

### Investigation of Electrically Controlled Drug Release Efficiency

The electrically controlled drug release efficiency of the fabricated MNs is investigated by placing the MNs in the agarose gel in vitro skin replica model, after giving the appropriate amount of current supply i.e., 0.5 mA for 5 mins. Then, the gels are incubated with 2 mL of methanol:water (1:1) for 12 hours at 37° C. with constant shaking and then sonicated for 10 mins. After that, the solutions are centrifuged, and the supernatant is analyzed by High-Performance Liquid Chromatography (HPLC, Agilent Technology). For the quantification of the Miconazole nitrate release from the MNs, an HPLC method is established using a C18 column and acetonitrile (ACN):methanol (9:1) as a mobile phase, 100 μL of the sample injection volume with a flow rate of 0.8 mL/min. For the comparison, M-MNs without electrical stimulation are considered as control.

Evaluation of Drug Penetration from Electroconductive MNs in an In Vitro Biofilm Model

The drug permeation characteristics of the electroconductive MNs are investigated using an in-vitro biofilm model. Briefly, Cy3-loaded MNs are placed onto the preformed biofilms of C. albicans (SC 5314) which expresses green fluorescent protein (GFP). The biofilm specimens are prepared by inoculating the GFP-Candida albicans (1×10⁶ cells/mL) onto the chamber slide for 3 days supplemented with yeast-peptone dextrose (YPD) media to form uniform thickness biofilms. Then the Cy3-loaded MNs are placed over the biofilm and constant current is applied for 5 mins by using a galvanic unit. After that, the MNs are removed and washed with 100 μL PBS to remove the unpenetrated dye and then the Cy3 distribution inside the biofilm matrix is evaluated by confocal imaging.

### In Vitro Biocompatibility Assessment of Electroconductive MNs

The biocompatibility of the fabricated MNs is evaluated against Human skin keratinocyte (HaCaT) cells using Cell Counting Kit-8 assay. In brief, HaCaT cells are cultured in Dulbecco's modified Eagle medium (DMEM) containing 10% Fetal Bovine Serum (FBS) and 1% Penicillin-Streptomycin and incubated in a humidified incubator (37° C., 5% CO₂). Then, 1×10⁵ cells are seeded in each well on a 24-well plate and incubated for 24 hours to form a monolayer. Simultaneously, the soak solutions of the M-MNs patch are prepared by submerging the MN patch inside the DMEM media for 24 hours. After that, 500 μl of medium containing soak solutions of M-MNs patch are added into the well and kept for 24-hour incubation at 37° C. After that, the media is removed and 300 μL of CCK-8 (Life Technologies) solution is added and incubated for 2 h in the dark. At last, the cell viability is determined by assessing the optical absorbance of each well using the CCK8 counting kit.

### In Vitro Antifungal Activity Assessment of Electroconductive MNs

The antimicrobial efficiency of the fabricated GelMA+CNT MNs is investigated using the CFU assay. For that, Candida albicans SC5314 (ATCC) species are grown overnight in the YPD broth at 37° C. in aerobic conditions. After that, 1×10⁶ cells are inoculated in 24 well plates and then the miconazole nitrate-loaded MNs are placed on the inoculum with and without current treatment. Untreated fungal cultures are taken as control. Then the treated plates are incubated in the dark at 37° C. for 4 hours, after that a 100-fold dilution is made from the culture solution and 10 μL solution is plated on the YPD agar plate using a spiral plater. Then the plates are incubated for 24 hours, and the colonies are counted and CFU values are calculated accordingly.

### Ex-Vivo Antifungal Activity Assessment of the Electroconductive MNs

The ex-vivo antifungal activity of the fabricated MNs is tested in a custom-designed porcine skin model. Briefly, skin specimens are shaved and hydrated in PBS (pH 7.4) for 1 hour, then cut into a rectangular shape with a dimension of 30 mm×20 mm, and soaked with 70% v/v ethanol for 30 mins. After that, 100 μL of Candida albicans suspension (6×10⁶ CFU/mL) will be injected subcutaneously into the skin. The inoculated skin specimens are placed on SDA plates for 3 days at 37-C to culture the inoculum. After three days' culture, different treatments are applied. Note that, the M-MN patches are applied with manual force for 30 seconds then a cylindrical stainless-steel weight (12 g) is placed on top of the patch to keep it in place. After 5 mins of application, skins are washed with PBS to remove the remaining drug, and after that, the skin specimens are collected using an 8 mm biopsy punch. Then, the specimens are homogenized with 1 mL of DMSO for 10 mins and centrifuged at 16,160×g for 20 mins to harvest all the fungi from the skin tissue. The resultant fungal suspensions are further diluted accordingly, and fungicidal activity is evaluated by counting the CFU (n=3) after 24 hours.

### In Vivo Infected Wound Healing Assay

In vivo performance of the antifungal drug-loaded electroconductive MNs is evaluated using a deep cutaneous fungal infection mice model. About 24 female C57BL/6J mice (6-8 weeks old) are taken for the study. About 5×10⁶ CFU/mL of C. albicans SC 5314 suspension is prepared in PBS, and 100 μL fungal suspension is injected intradermally into the shaved back of each mouse. After 1-day, each of the mice develops inflamed nodules, indicating the successful deep fungi infection development.

After that, all the animals are divided into four groups randomly and denoted as control, cream, M-MNs and M-MNs+current group. On Day 3 and Day 5, the respective treatment is given to all mice except the untreated control one. For the visual confirmation and nodule area reduction, digital images are taken. On Day 6, all the animals are euthanized, and skin samples are harvested for fungal burden, histological, and immunohistological analysis. Furthermore, blood and different organ specimens namely, kidney, heart and spleen are also collected to confirm the biocompatibility of the electroconductive MNs.

### Histological and Immunohistological Study

For the histological and immunohistological analysis skin tissue specimens are collected in 10% formalin solution left for a day at room temperature and subsequently immersed into 30% sucrose solution for two days. After the stipulated time samples will be frozen which are then cut into slices of 5 μm thickness. For the CFU counting, fresh infected skin and blood specimens are homogenized using a tissue homogenizer for 15 mins and then centrifuged with PBS to collect the fungi and 10 μL of the suspension are cultured in YPD agar plates. The antifungal and tissue healing activity of the electroconductive MNs are assessed by Hematoxylin and Eosin, Periodic Acid Schiff (PAS), Masson's trichrome, and immunohistochemical staining.

### Statistical Analysis

All data in this work are presented as mean±standard deviation (SD). Statistical analysis of data is performed by the unpaired Student's t-test and one-way ANOVA among multiple groups. Immunofluorescence and histological images are quantified using Image-J (National Institutes of Health, Bethesda, Maryland, USA). Statistical analysis is performed with the GraphPad Prism v 8.5 software. *P<0.05, **P<0.01, ***P<0.001, and ****P<0.0001 are considered statistically significant.

### Example 2

### Fabrication of Electroconductive MN Patch

To create a highly electroconductive MN patch that can electric-assisted deliver the drug molecules in the subcutaneous layer by overcoming the dermal tissue barrier, excellent biocompatible GelMA polymer reinforced with highly electroconductive CNT composite is precisely selected to fabricate the electroconductive MNs.

The electroconductive MNs are fabricated by following the two-step micro-molding process. Turning to FIG. 1A, the present invention has chemically modified naturally derived gelatin with the methacrylic anhydride in the presence of Irgacure 2959 crosslinker under the UV radiation (365 nm) to synthesize photo-crosslinkable GelMA molecule. GelMA is synthesized as follows: 10 g of gelatin is dissolved in 100 mL of PBS at 50° C. Then, 8 mL of methacrylic anhydride is added dropwise and the solution is kept constant stirring at 60° C. for 3 h. Subsequently, 400 mL of PBS is further added to the solution and stopped the reaction. The resulting solution is dialyzed against deionized (DI) water at 40-50° C. for 7 days to remove unreacted methacrylic anhydride and other impurities. Lastly, the GelMA is obtained through lyophilization.

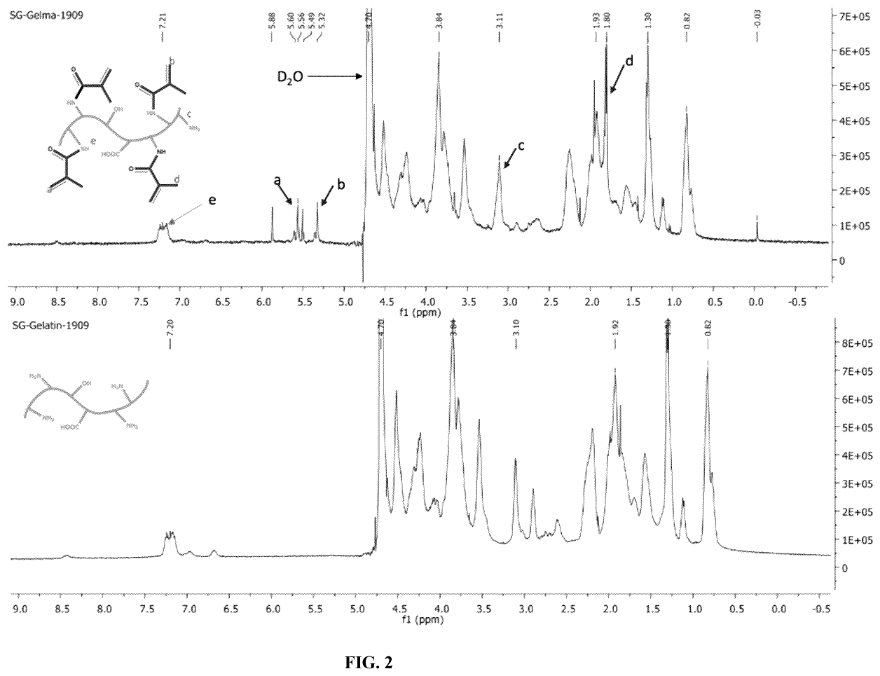

The resulting GelMA is characterized by proton nuclear magnetic resonance (¹H-NMR) spectroscopy, as shown in FIG. 2. Peaks observed at chemical shifts of 5.2 ppm and 5.8 ppm confirm methacrylic substitution on lysine residues in the gelatin backbone. Additionally, by calculating the change in the intensity of the phenylalanine peak, presence at 7.24 ppm, the degree of methacrylic substitution to be 53.4%, which is extremely suitable for photo-crosslinking.

After that, CNT is incorporated into the GelMA matrix during the fabrication of MN patches, owing to its excellent electrical conductivity and large surface area for drug loading. 100 mg/mL of aqueous GelMA is thoroughly mixed with CNT at the pre-designed concentration (i.e., 0, 2.5, 5.0, or 10.0 mg/mL). The crosslinker, Irgacure 2959, is added to the mixture at a concentration suitable for UV photo-crosslinking.

A polydimethylsiloxane (PDMS)-based negative mold is developed using a pyramidal MN stainless steel mold consisting of 100 needles in a 10×10 array with a height of 700 μm, tip radius of 5 μm and a base width of 300 μm (Micropoint Technologies Pvt. Ltd., Singapore). After that, the negative mold is prepared by pouring PDMS (10:1 w/w ratio as a curing agent) prepolymer solution over the stainless-steel mold and placed in a vacuum for degassing. Then, the mold is heated at 70° C. for 2 hours and peeled off. The obtained PDMS-micro mold is then repeatedly used for the fabrication of electroconductive MNs.

Next, the micromolding technique is used to fabricate the MN patches with a uniform shape (i.e., 10 mm×10 mm). The GelMA mixture is poured into the PDMS-based negative mold containing inverted conical cavities. To ensure uniform distribution and remove air bubbles, the mold is subjected to centrifugation at a speed of 4,000 rpm for 5 mins. The excess solution remaining on the mold surface is removed using a glass slide. Then, a second layer of 0.5 g·mL⁻¹ of GelMA+CNT dispersion is added to create the back layer of the MN patch. The mold is exposed to UV light (365 nm) for 10 minutes to initiate the photo-crosslinking reaction, solidifying the GelMA and forming the MN array. The crosslinked MN patches are air-dried and carefully peeled off from the PDMS mold, resulting in sharp and uniform microneedle structures. The fabricated MN patches can be easily stored more than 180 days in a dark, dry-place without any significant loss of therapeutic activity.

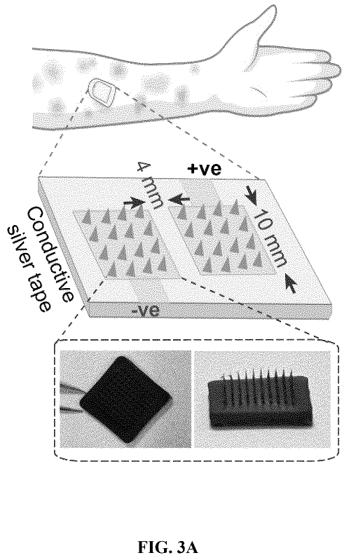

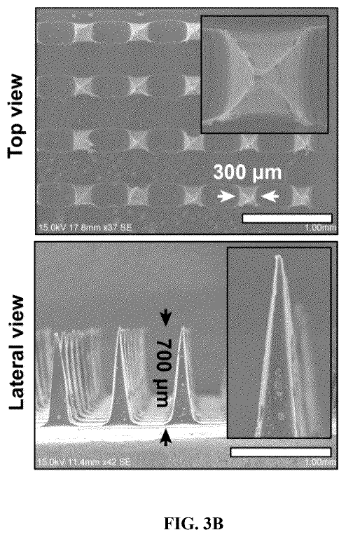

For the electricity supply, two separate MN arrays are attached on the silver adhesive tape at a 4 mm distance and connected to external power source with silver wires. FIG. 3A represents the dimensions of individual MNs array with the digital image.

FIG. 3B represents the SEM images of the fabricated MNs, which have a sharp-pointed pyramidal shape with a uniform diameter of 300 μm and a length of 70 μm.

To create drug-loaded electroconductive MNs, 1 mg/mL of miconazole nitrate (equivalent to 400 g per patch) is incorporated into the GelMA+CNT dispersion.

### Example 3

### Characterization of the Electroconductive MN Patch

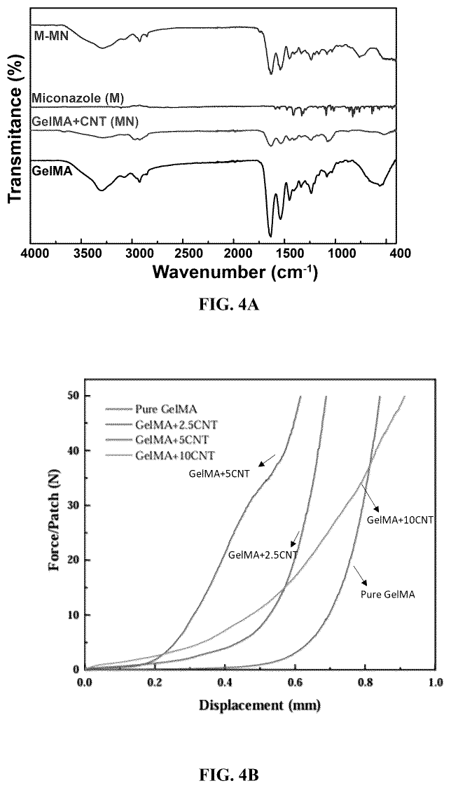

To confirm the successful reinforcement of CNT into the GelMA matrix, the GelMA+CNT composite is further characterized after photo-crosslinking using Fourier transform infrared (FTIR) spectroscopy. As shown in FIG. 4A, pure GelMA has a C═O vibration peak, C—N stretching vibration peak, and —NH stretching vibration peak. The —OH stretching peak appears at 995, 1430, and 1150 cm⁻¹, respectively. Similarly, in the GelMA+CNT composite, all the above peaks are presented and slightly shifted due to the H-bonding and π-π interaction between the delocalized π-electrons presented on the CNT surface and the aromatic ring presented in the GelMA polymer. These results suggest that, following the successful reinforcement of CNT into the GelMA matrix, the chemical characteristics of the individual components are still preserved.

In addition, the present invention also investigates the mechanical strength of the fabricated MN patch to ensure its piercing capability into the dermal tissue layers. To achieve the optimized mechanical strength, different concentrations of CNT are reinforced, including low (1-2.5 wt %), medium (3.0-5.0 wt %), and high (7.5-10.0 wt %) concentrations, with pristine GelMA, and then the MNs are prepared. The mechanical strength of the MNs is evaluated using a compression test compared to pristine GelMA. FIG. 4B indicates that as the amount of CNT reinforcement increases, the deformation of the fabricated MN patch decreases, thereby enhancing the mechanical properties of the MNs. This enhanced mechanical strength can be attributed to the micro/nano-sized CNTs (e.g., 5-10 nm), which act as nanofibers to reinforce the polymer composite, enabling uniform stress distribution and resisting crack propagation within the polymer matrix. However, when the CNT content reaches 10 wt %, the mechanical strength of the MNs significantly decreases significantly, possibly due to the agglomeration of CNTs inside the GelMA matrix. Compared to pristine GelMA, both the GelMA+2.5 CNT and GelMA+5.0 CNT MNs demonstrate improved mechanical properties than pristine GelMA, where all the microneedles are withstanding 0.5 N/needle i.e., 50 N force per patch without any deformation, which is adequate for efficient penetration into the human skin.

Taken together, these results confirm that the electroconductive MNs can easily penetrate the human dermal barrier to deliver the intended therapeutic agents. In particular, only GelMA+2.5 CNT and GelMA+5.0 CNT are suitable for effectively penetrating the human dermal barrier to deliver the intended therapeutic agents (FIG. 5). The precise combination of these two materials in specific ratios makes the system unique. If the ratios and amounts of the substances are modified, the entire electrical properties of the electroconductive MNs would change, and as a result, the therapeutic outcomes would also differ.

The electrical properties of the electroconductive MN patches are examined by measuring their impedance value using a handheld digital multimeter. The electrical conductivity of the fabricated MNs is evaluated by comparing the impedance value of each MN.

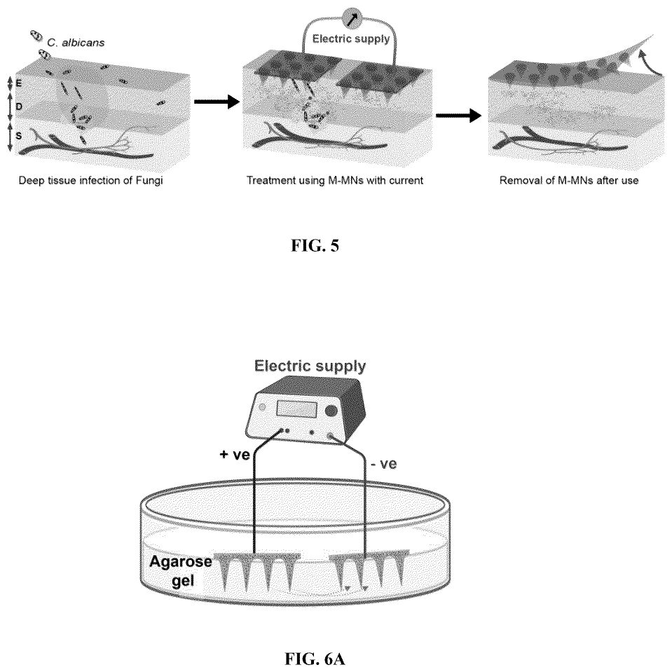

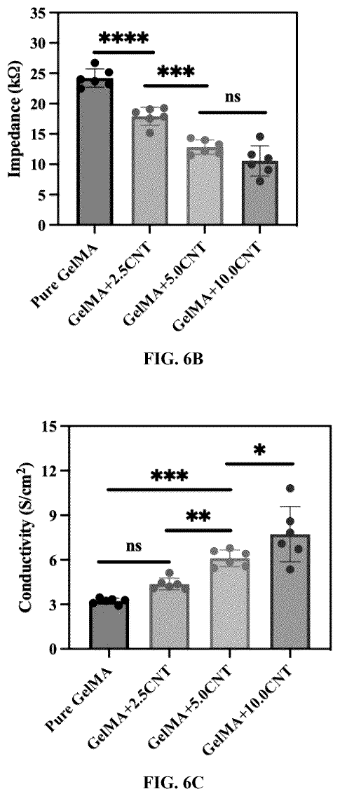

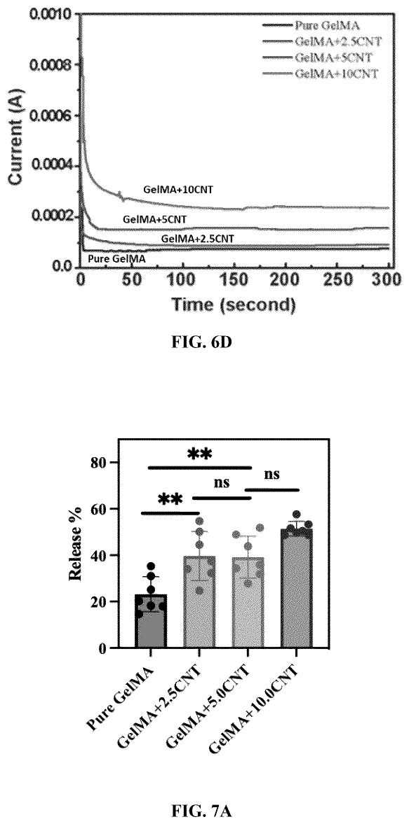

FIG. 6A illustrates a setup for impedance measurement. In this test, 1.4 wt % agarose gel is used as an in vitro replica model since it possesses electrical properties similar to those of human skin tissue. Turning to FIG. 6B, the impedance of the MN patch decreases significantly with the addition of CNT, compared to pure GelMA MNs. However, the impedance between GelMA+5.0 CNT and GelMA+10.0 CNT remains statistically insignificant. The electrical conductivity of the developed electroconductive patch calculated based on the impedance of the MN patches shows that GelMA+10.0 CNT has the highest electrical conductivity among all the fabricated MNs (FIG. 6C). However, it has poor mechanical strength than GelMA+5.0 CNT, and therefore GelMA+5.0 CNT (i.e., 5.0% CNT) is selected as the optimized composition for developing electroconductive MNs.

Furthermore, the electrochemical stability of the fabricated electroconductive MNs is also tested using an electrochemical workstation, by placing the MNs on an agarose gel. To assess the electrochemical stability of the electroconductive MNs, a constant 1V voltage difference between the two electroconductive MNs is applied, and the output current is monitored (FIG. 6D). CNT reinforcement not only increased the output current but also enhanced it for all GelMA+CNT MNs. The results also suggest that the incorporation of CNT leads to an increase in the output current, as the presence of CNT reduced the overall resistance of the material. More importantly, the current flow remains constant throughout the tested period, indicating the stable electroconductive performance of the MN patches.

### Example 4

### Drug Delivery Performance of the Electroconductive MN Patch

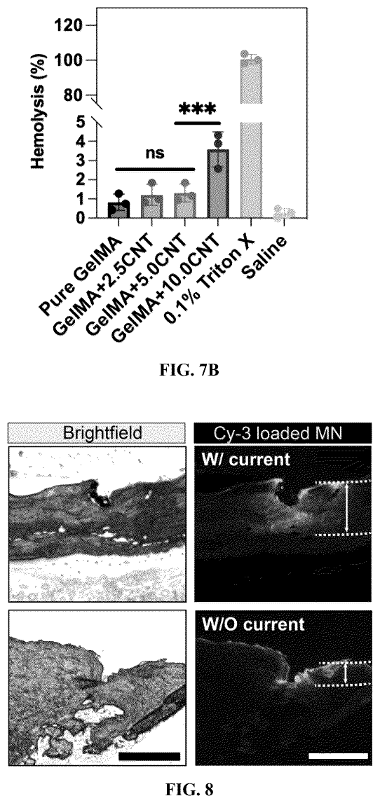

To evaluate the electrically controlled drug release performance of the electroconductive MNs, the release of Cy3 dye under electric current stimulation is monitored. The Cy3-loaded electroconductive MNs patches are placed on the agarose gel (1.4 wt %), and 0.5 mA current is supplied for 5 mins using Galvanic cells. Immediately after the insertion, the MNs are observed to swell and undergo burst release upon electric stimulation. After that, the amount of Cy3 released is quantified through fluorescent spectroscopy. As shown in FIG. 7A, the percentage of Cy3 released significantly increases from 20% in pure GelMA group to around 40%-50% in CNT-incorporated GelMA group. This result confirms that the increase in the net electrical conductivity of the electroconductive MNs enhances the drug release under the same current application. However, there is no significant difference in terms of drug release among different groups of GelMA with CNT. Collectively, the results confirm the addition of CNT contribute to increased net electrical conductivity of the MN patches, which is beneficial for electric-driven drug release.

The tissue microenvironment of the infected wound can trigger rapid bleeding, which can washout the drug molecules from the site of infection and the blood coagulation after the MN contact also leads to worsening the infection. Therefore, to further investigate the potential of electroconductive MNs as a treatment for fungal infection, the blood compatibility is examined according to ISO 10993-4:2017 standard. As observed in FIG. 7B, up to 5.0% CNT reinforcement MNs, (i.e., GelMA+2.5/5.0 CNT) the % hemolysis is below the 2% which is clinically acceptable, however in the case of 10% CNT reinforced MNs i.e., GelMA+10.0 CNT exhibits around 4.8% hemolysis which is significantly higher. Thus, the present invention demonstrates that the MNs composed of GelMA+5.0 CNT exhibit excellent electroconductivity, along with sufficient mechanical strength and hemocompatibility, making them capable of delivering antifungal molecules to deeply infected tissue environments. Considering that the GelMA+5.0 CNT MN patch exhibits comparable electrical conductivity and drug release efficiency to the GelMA+10.0 CNT treatment group, while offering superior mechanical strength and biocompatibility, it is selected for further investigation.

### Example 5

### Ex Vivo and In Vivo Evaluation of Electroconductive MN Patch

The capability of penetrating skin tissue and delivering drug is tested using porcine ear skin as an ex vivo model. A fresh porcine cadaver ear model is utilized to test skin penetration characteristics of the MNs. Briefly, Cy3-loaded MN patch is pressed into the porcine ear skin with approximately 1.5 N of force for 1 min. Next, the tissue samples with MNs inserted are freshly frozen in Optimum Cutting Temperature (OCT) liquid, and 5 μm thick cross-sectional slices are prepared by microtome and then visualized on the microscope.

Referring to FIG. 8, a noticeable indentation is observed in the epidermal layer of porcine skin following the application of electroconductive MN patches. However, the diffusion of Cy3 into the dermal layer of porcine skin became apparent only after the application of electric stimulation.

Furthermore, in the in vivo system, this passive diffusion often results in the drug permeating blood vessels, leading to systemic toxicity due to the potent antifungal agents. On the other hand, the electroconductive MNs, when combined with electric current stimulation, enable precise localization of the drug molecules at the periphery of the applied tissue, and avoid toxic complications. These results confirm that the electroconductive MN patch can effectively deliver therapeutic molecules into deep cutaneous tissue assisted by electric stimulation.

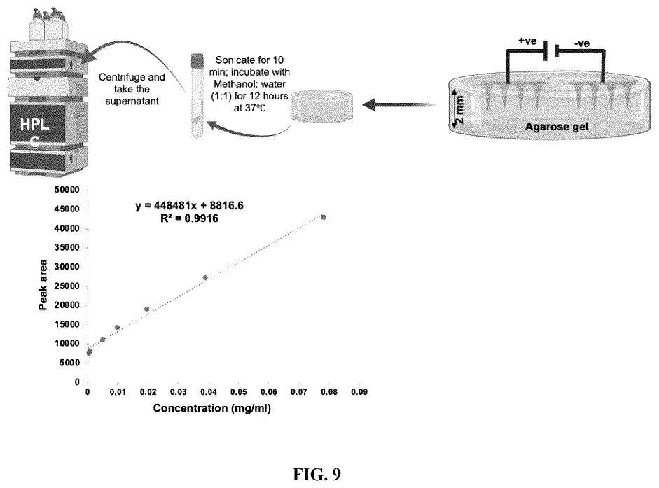

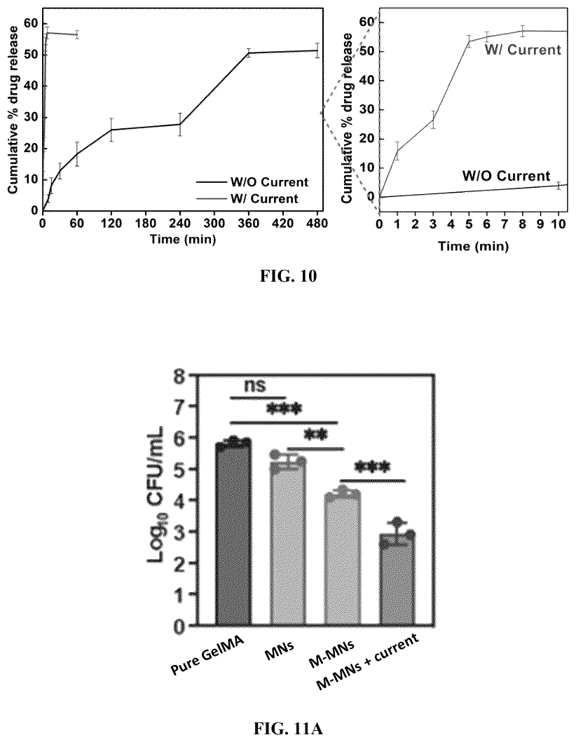

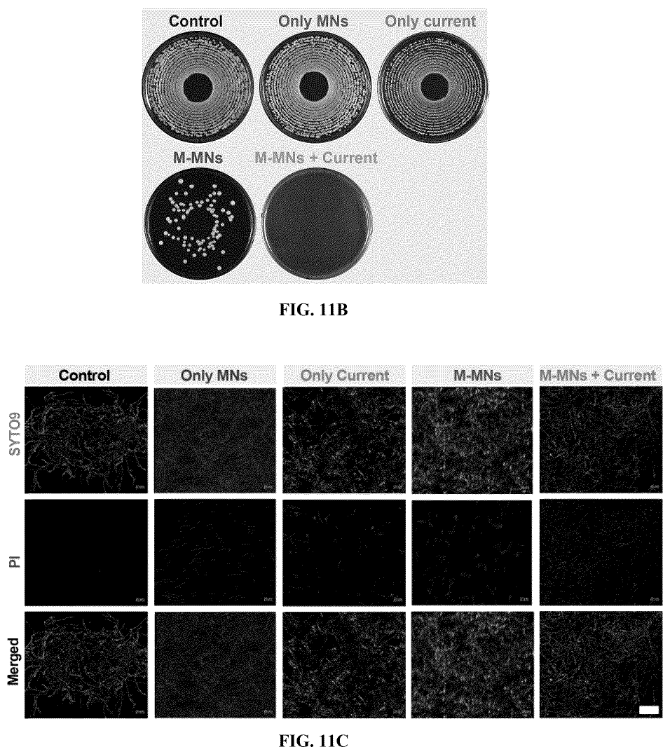

To explore the potential of the electroconductive MN patch for anti-fungal therapy, miconazole nitrate, a well-known first line antifungal agent for treating cutaneous fungal infection, is loaded into MNs. The miconazole-loaded MN patch is then placed on 1.4 wt % agarose gel and given a current of 0.5 mA for 5 mins. The drug release is determined by analyzing the amount of miconazole recovered from the gel using HPLC (FIG. 9). The data show that over 55% of the loaded drug (approximately 224.3 g) is released within 5 minutes, while the non-electrically stimulated MN patch takes more than 360 minutes to release 50% of the drug (FIG. 10). The results have confirmed the potential of the electroconductive MN patch for on-demand drug release driven by electrical stimulation.

### Example 6

### In Vitro and Ex-Vivo Antifungal Activity of M-MNs

To test the antifungal efficacy of the M-MNs, Candida albicans SC5314 strain is selected as a representative fungi microorganism due to its rapid yeast-to-hyphae transition, which may lead to systemic invasive deep-tissue fungal infection.

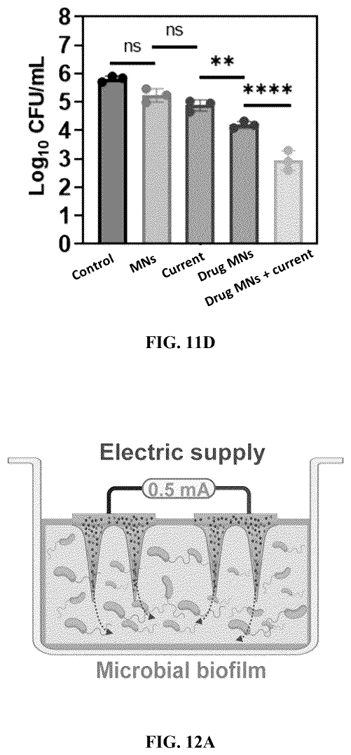

The antifungal activity of the M-MNs is tested against both planktonic and biofilm microenvironments. Turning to FIGS. 11A-11B, the effects of electroconductive M-MNs on planktonic C. albicans cells are evaluated. The CFU counting reveals a 2.5 log reduction, indicating that 99% of C. albicans is eliminated. In contrast, M-MNs without electrical stimulation only achieve a reduction of around 1.2 logs in CFU. Similar trends are observed in the live/dead staining assay images, where the number of dead cells is significantly higher in the MNs+current treatment group compared to other treatments group (FIG. 11C). These results provide direct evidence that the M-MNs effectively exhibit antifungal properties. To exclude the possibility of direct fungicidal effect of applied electrical stimulation, it is also found that applying only 0.5 mA current to non-drug loaded MNs does not elicit any significant antifungal effect (FIG. 11D).

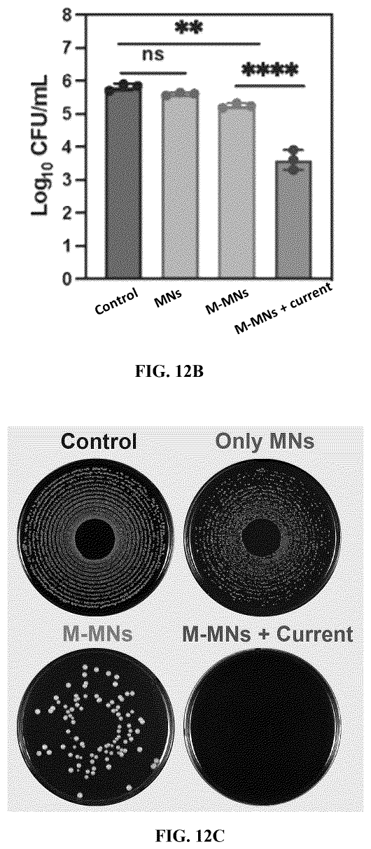

The development of microbial biofilm is a major factor contributing to the failure of conventional therapeutic systems in treating fungal infections. The dense extracellular matrix of fungal cells acts as a barrier to drug permeation, leading to the formation of persister cells. Therefore, the present invention also investigated the drug permeation and biofilm eradication ability of the electroconductive MNs against C. albicans biofilm, presented in FIGS. 12A-12C. The effectiveness of M-MNs on preformed C. albicans biofilm is investigated. M-MNs alone achieves only a 0.5 log reduction in CFU, whereas M-MNs with electric current demonstrates a significantly improved therapeutic effect, eliminating approximately 99.9% of the biofilm (around a 2.5 log reduction).

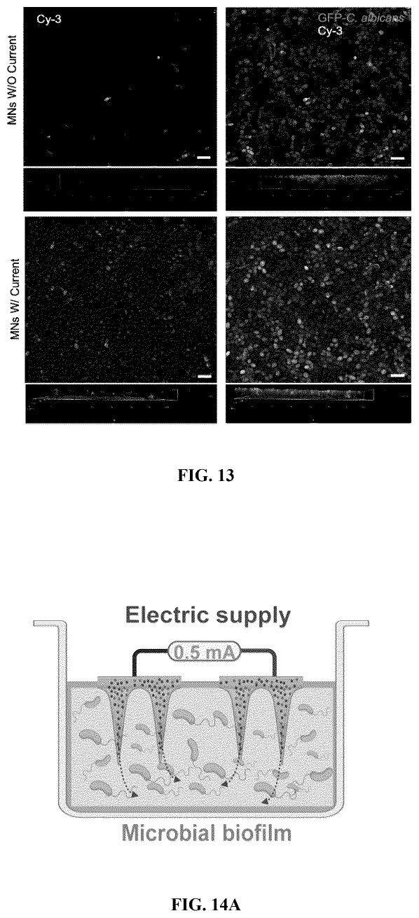

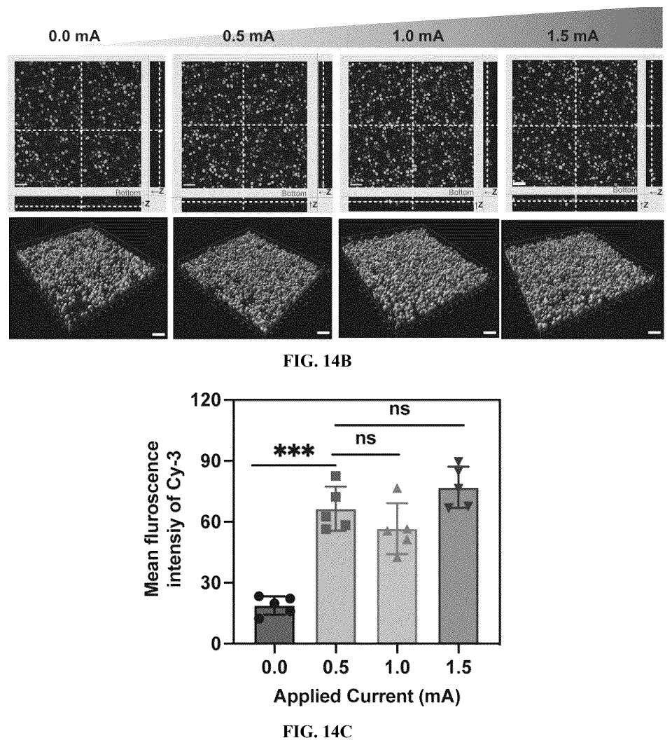

Furthermore, to examine the drug penetration ability of the electroconductive MNs inside the biofilm matrices upon electric supply more accurately, 3 days preformed in vitro biofilm in the chamber slides with GFP-expressing C. albicans is developed. The Cy3 molecules (0.5 mg/mL) are loaded into the electroconductive MNs as a model drug. Using confocal microscopy, FIG. 13 shows the CLSM images of the Cy3 penetration from the MNs+current treatment into the biofilm following MNs+current treatment, with a 0.5 mA current applied for 5 minutes. As presented, it is clearly observed that the application of MNs+current not only delivers a high amount of drug (i.e., Cy3) into the biofilm matrices but also helps to penetrate the drug molecules deeper, as evidenced by the vertical cross-section images. Nonetheless, the variation in drug penetration with different current supplies over a fixed time period (i.e., 5 minutes) is also examined. FIGS. 14A-14C shows that further increasing the current intensity does not lead to a higher amount of Cy3 release. However, for the current study, the present invention optimizes the current supply to a minimal level i.e., 0.5 mA for 5 mins, as this amount of current density does not exhibit any external tissue damage, and also from the drug release study, the present invention confirms that the 0.5 mA current supply for 5 mins can release 53% (i.e., 212 g) of the loaded drug molecules (i.e, miconazole nitrate) which is sufficient for the fungicidal effect. Taken together, the results confirm that the M-MNs with a 0.5 mA current supply for 5 minutes significantly demonstrate both fungicidal and biofilm eradication abilities.

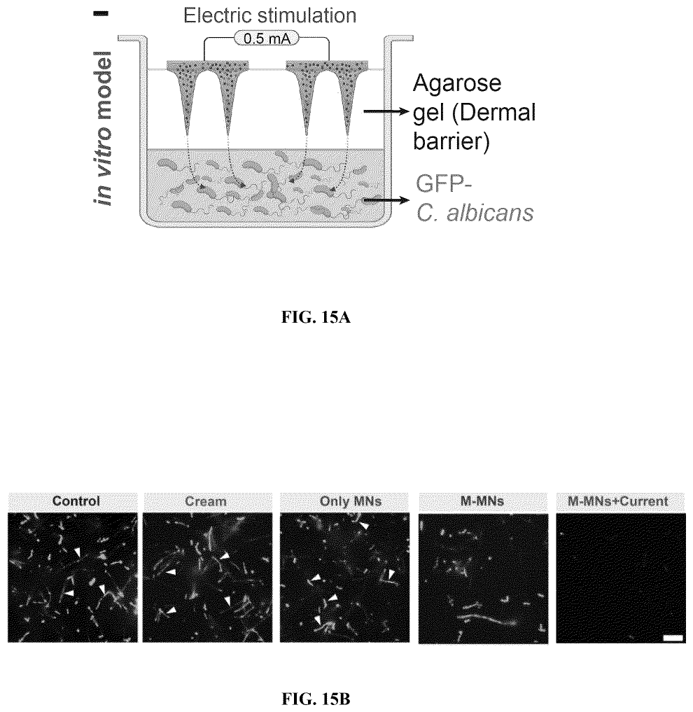

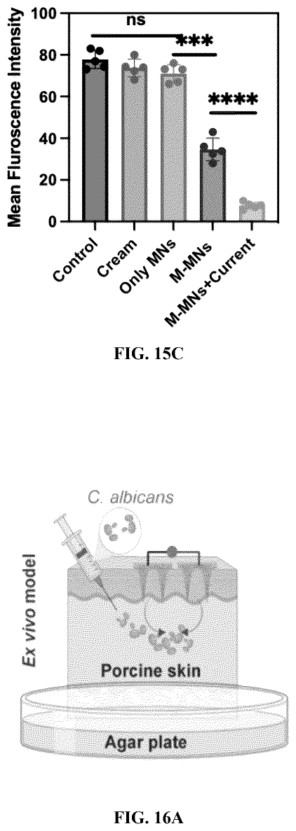

In the clinical setting, microbial cells can breach the dermal barrier and translocate to the deeper cutaneous layers, where they form biofilms that are more challenging to eradicate. Previous data have demonstrated that electrical stimulation can facilitate the delivery of Cy3 to deeper layers of biofilm. This example further investigates whether electrically-driven M-MNs can be used to eliminate deep cutaneous infections by targeting pre-formed C. albicans biofilms located beneath a 2 mm-thick agarose gel. The schematic is shown in FIG. 15A. To get more insight about the efficacy of the fabricated electroconductive MNs, the biofilm eradication ability is tested and compared with conventional fist-line of treatment of cutaneous fungal infection such as, commercially available topical 2% miconazole nitrate cream. FIG. 15B represents the confocal images of the fungal biofilm after being treated with different formulations on the in vitro model. The quantitative analysis of the biofilm fluorescence intensity (FIG. 15C) reveals that, when compared to the control, the cream does not cause any reduction in the biofilm matrices. On the other hand, both the M-MNs and M-MNs+current groups show a clear reduction in the biofilm. This is because most conventional topical cream/gel formulations are unable to penetrate deep tissue layers (with drug diffusion typically limited to the 50-500 μm range). In contrast, conventional MNs can overcome this issue, but the amount of drug diffusion remains at subtherapeutic levels, insufficient to eradicate the entire fungal burden⁹. Therefore, the electroconductive MNs+current treatment can effectively overcome these limitations by delivering antifungal drug molecules to deeper layers through an on-demand burst release mechanism.

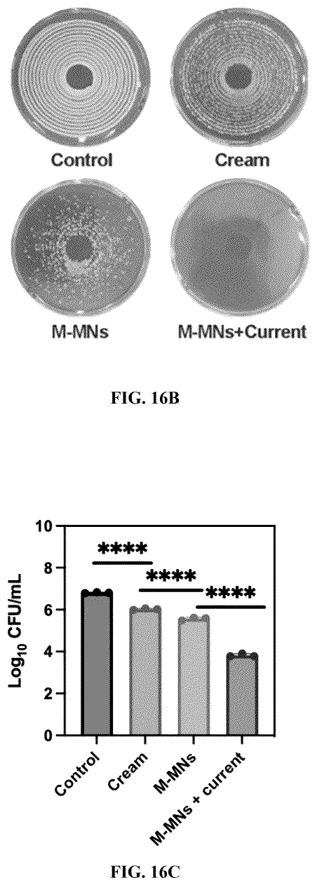

To further investigate the antifungal properties of the electroconductive MNs, an ex vivo experiment is conducted using a porcine skin model. An intradermal solution of C. albicans is injected into a fresh porcine skin specimen and inoculated it on the Sabouraud dextrose agar (SDA) plate for about three days. The schematic is shown in FIG. 16A. Subsequently, different treatments are applied, and homogenized tissue samples are collected for CFU counting to assess the remaining fungal burden. FIGS. 16B-16C demonstrate the fungicidal activity of the M-MNs in the ex vivo study. The results suggest that miconazole cream and M-MNs alone lead to a respective reduction of 0.5 and 1 log in CFU compared to the untreatment group. The reduced CFU count in the MN groups, compared to the cream treatment group, further confirms the limited effectiveness of the conventional MN system. In contrast, the MNs+current treatment group shows an approximate 2.5 log reduction in CFU (i.e., >99.9%) compared to both the MNs and cream treatment groups. These results further confirm that the electroconductive MNs effectively eliminate deep cutaneous fungal infections by delivering therapeutic levels of antifungal molecules to the deeper tissue layers.

### Example 7

### In-Vitro Biocompatibility of the Electroconductive MNs Patch

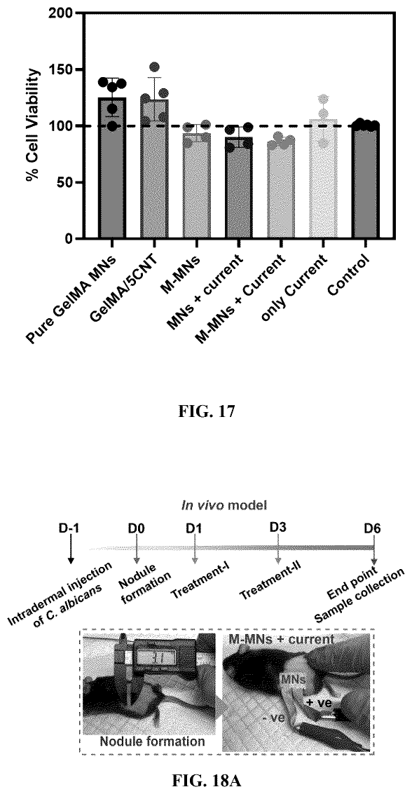

In this example, a traditional cell viability assay is done against HaCaT skin keratinocyte cells. As shown in FIG. 17, none of the tested materials, including pure GelMA, GelMA incorporated with CNT, and miconazole-loaded MNs, display any signs of cytotoxicity. More than 90% of the cells remained viable in all groups, including the MNs+current treatment group, confirming the biocompatibility of the fabricated electroconductive MNs. Nonetheless, it is also evidenced that mild external electric stimulation favors cell proliferation and accelerates the wound-healing process, which also reconfirms the efficacy of the fabricated electroconductive MNs¹⁰. Furthermore, the applied current does not compromise the biocompatibility of the electroconductive MNs.

### Example 8

### In Vivo Evaluation of Electroconductive MNs for Treating Deep Tissue Infection

Deep cutaneous infection is considered as more fatal clinical condition than the superficial infections due to its lethality and prone to develop systemic infection. Specifically, fungal species are more difficult to remove than bacteria, as their eukaryotic cellular structure with thick, stiff cell walls that resist to innate immune system components. Therefore, the eradication of fungal cells within deep tissue layers has long been a clinical challenge.

In this example, in vivo therapeutic efficiency of the electroconductive MNs on deep cutaneous fungal infection is investigated using a C. albicans inoculated subcutaneous infection model.

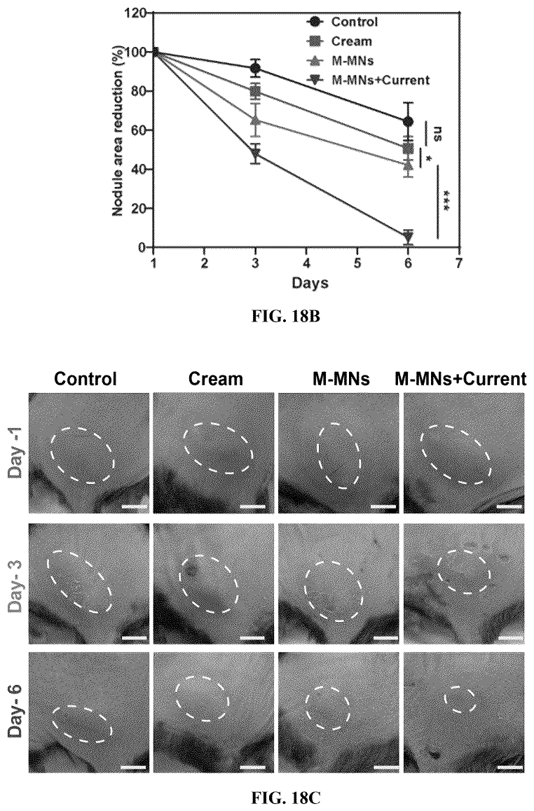

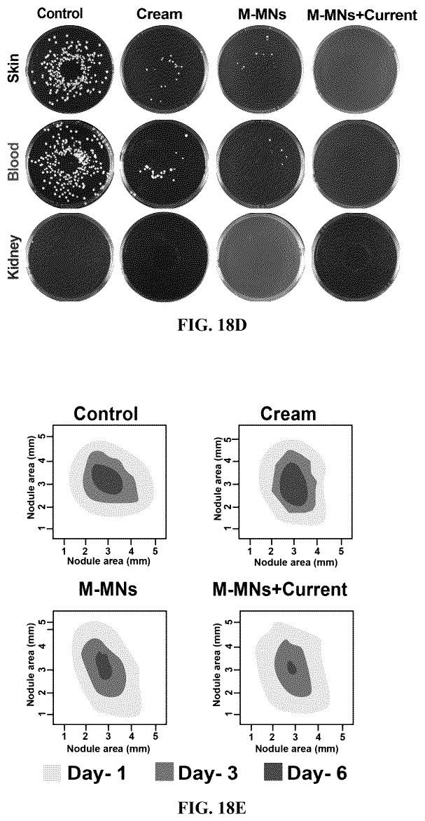

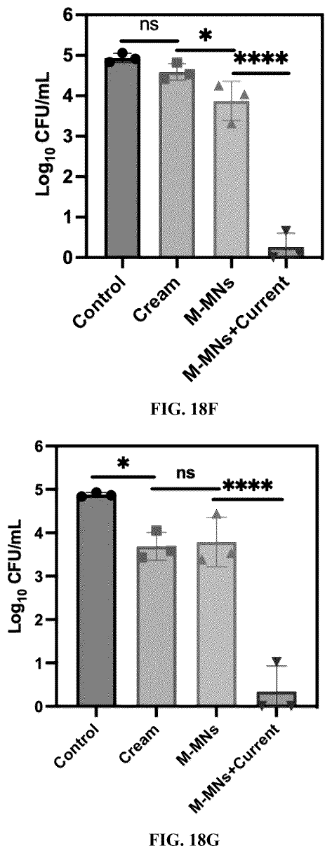

Referring to FIG. 18A, at day 1, C. albicans SC5314 suspension is injected intradermally, and on day 1 and day 3 after the induction of infection, the mice are separated into four different groups i.e., (1) Untreated control, (2) Cream group (i.e., commercially available 2% miconazole nitrate cream, Zarin™), (3) M-MNs group, and (4) M-MNs+current group, randomly (n=6). The progression of subcutaneous fungal infection is monitored by the size of nodule on the dorsal skin. After day 1, Prominent nodules form under the skin, confirming the successful establishment of the cutaneous fungal infection model. On day 1, the nodules of all the mice treated with cream, M-MNs, and M-MNs+current, and the non-treated mice serve as control. As time progresses, a significant reduction in nodule area is observed in all the MN treatment groups. For example, by day 6 after nodule formation, all four groups exhibit a significant decrease in nodule size. However, compared to the 35%-45% reduction in the control and cream treatment groups, M-MNs alone contribute to an approximately 60% reduction.

FIGS. 18B-18C indicate that the nodule area in the M-MNs+current group is reduced to almost invisible, compared to the cream and M-MNs group, which show approximately 22%, and approximately 35% nodule area reduction respectively. When facilitated by electric current, the nodule size is further reduced to around 5% of their original size.

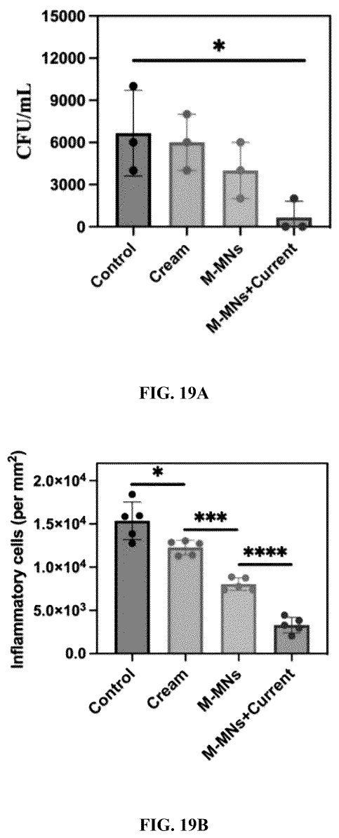

Moreover, the fungal burdens in skin, blood and kidney are further determined by CFU assay. On day 6, all the mice are euthanized and the skin samples with the nodules are collected. As shown in FIGS. 18D-18G, both in the skin and blood specimens from the M-MNs+current treatment groups exhibit a significant reduction in CFU (approximately 3-fold log reduction) compared to all other treatments. However, it is important to note that neither method completely eradicates the cutaneous fungal infection nor prevents systemic infection. It has also been observed that, compared to topical cream application, the MN treatment reduces the fungal burden but is not capable of completely eradicating the fungal infection. This is because the MNs are capable of delivering the drug to the deep cutaneous infection site, but their conventional release kinetics limit the amount of drug released. As a result, the subtherapeutic drug levels are insufficient to effectively treat the deep cutaneous infection. Thus, in these two groups, it is able to detect C. albicans with CFU assay in the kidney of mice (FIGS. 18D and 19A). Whereas, electroconductive M-MNs demonstrate superior antifungal effects, the fungal burdens in skin and blood samples are dramatically decreased by more than 4-log CFU. It is only able to detect a minimal amount C. albicans in kidney by CFU assay from 2 out of 3 mice treated by electrically driven M-MNs.

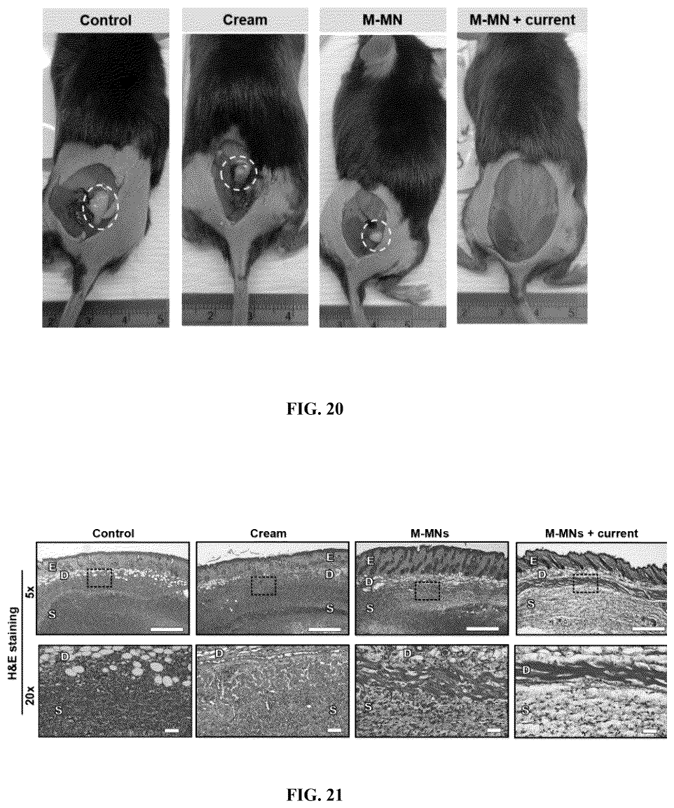

Interestingly, as depicted in FIG. 20, pus-like debris is present beneath the nodule in the control, cream, and MNs treatment groups, whereas it is absent in the M-MNs+current treatment groups. This observation provides direct visual confirmation of the electroconductive MNs' efficient capability to eradicate fungal infections.

To further investigate the biocompatibility and the therapeutic effect of the electroconductive MNs, skin and organ specimens are also collected for histological study using the HE staining. The subcutaneous lesions and the micro-architecture distortion are prominent in the control, cream-treated and M-MNs groups. However, in the group treated with M-MNs and electrical stimulation, the histological morphology of subcutaneous tissue is almost restored to the healthy state. Turning to FIG. 21, the HE staining shows that immune responses, as evidenced by the presence of inflammatory cells, are significantly higher in the control and cream groups compared to the M-MNs and M-MNs+current treatment groups. Referring to FIG. 19B, a large number of inflammatory cells are present in the subcutaneous layer of non-treated mice. Treatment with the cream reduces the number of inflammatory cells by approximately 16%, while the MNs treatment decreases their number by 50%. With the application of electrical stimulation, the number of inflammatory cells decreases by more than 83%. Additionally, the thickness and micro-architecture of the skin in the M-MNs+current treatment groups closely resemble those of healthy skin, whereas the cream and M-MNs groups still exhibit notable infection and swelling.

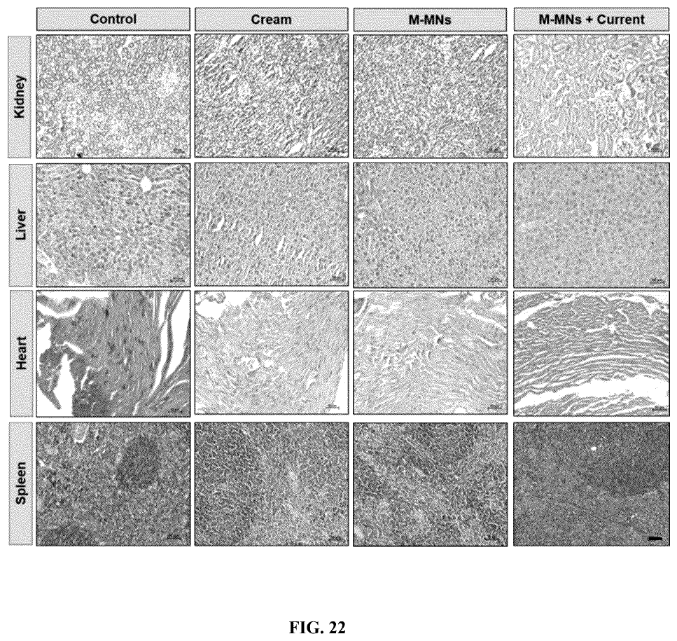

Moreover, the biosafety of the electroconductive MNs is also confirmed by evaluating the histology of different organs such as the kidney, heart, liver, and spleen, using HE staining. As depicted in FIG. 22, the biosafety of electroconductive MNs is further confirmed by the absence of significant histological alterations in major organs, including the kidney, heart, liver, and spleen. This demonstrates that the MNs+current treatment is a completely safe approach for combating cutaneous infections.

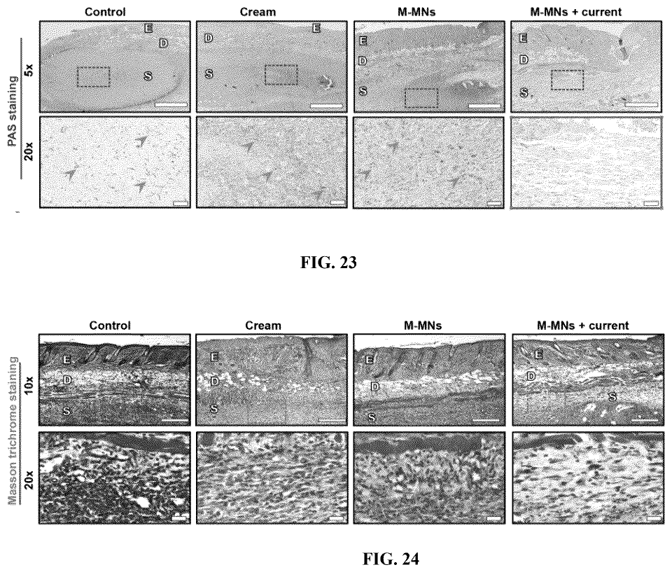

Next, Periodic Acid Staining (PAS), a widely used strategy for visualizing polysaccharides specifically presented on the fungal cell wall, is performed to assess the antifungal efficiency of the electroconductive M-MNs. As presented in FIG. 23, even after the treatment of cream or M-MNs on day 6, a large number of fungal cells remain in the cream and M-MNs groups. In contrast, the electrically-driven M-MNs treatment group only shows traces of dead fungal cells. These results provide compelling evidence for the effectiveness of electroconductive M-MNs in treating deep cutaneous fungal infections.

### Example 9

Effect of Electroconductive M-MN Patch on Collagen Regeneration and Wound Healing after Fungal Infection Removal

In this example, the effect of the electroconductive MNs on healing cutaneous tissues after the infection removal is further investigated. For that, Masson trichrome staining is performed to elucidate the microarchitecture of the infection site and assess the collagen content. Prior studies have reported that the transient amount of electricity supply stimulated fibroblasts and myofibroblast cells residing in the wound site to induce collagen synthesis which subsequently accelerated the wound-healing process¹⁰. Similarly, the image in FIG. 24 shows a greater amount of regenerated collagen fibers in the subcutaneous layer of the skin treated with electroconductive M-MNs, compared to the cream and M-MNs treatment groups. Compared with the cream and M-MNs groups, a high amount of collagen is observed in the M-MNs+current group. This observation directly proves the wound-healing potential of the electroconductive MNs after the eradication of the fungal infection. It can stimulate fibroblasts and myofibroblasts residing within the injured area to accelerate collagen synthesis.

### Example 10

### Electroconductive MNs Drive Neuro-Immune Crosstalk for Healing Infected Wounds

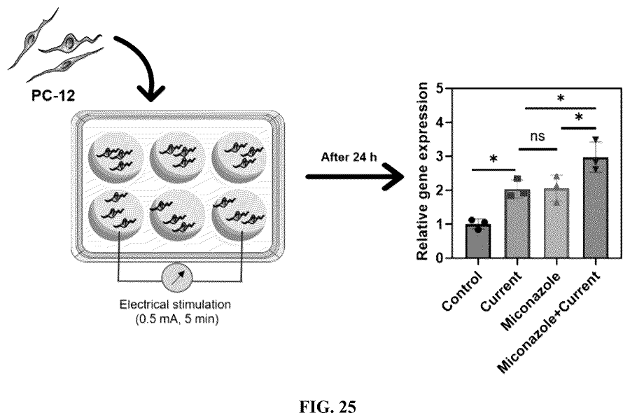

It is hypothesized that the miconazole nitrate molecules released from the electroconductive MNs may also trigger the release of the calcitonin gene-related peptide (CGRP) from nociceptive neurons under electrical stimulation. The presence of CGRP+sensory neurons in the subcutaneous layer after treatment is investigated, as both miconazole and electrical stimulation are known to promote axonal growth and neuronal activation. The present invention demonstrates that the electroconductive MNs under the low-voltage current stimulation activate the sensory neurons present in >1500 μm deep tissue. FIG. 25 uses PC-12, a widely used neuronal cell line, and demonstrates that miconazole and electric current synergistically contribute to a nearly 3-fold increase in the expression of CGRP, which is more effective than either treatment alone. This observation correlates with previous studies in which CGRP expression is enhanced following electrical current stimulation¹¹.

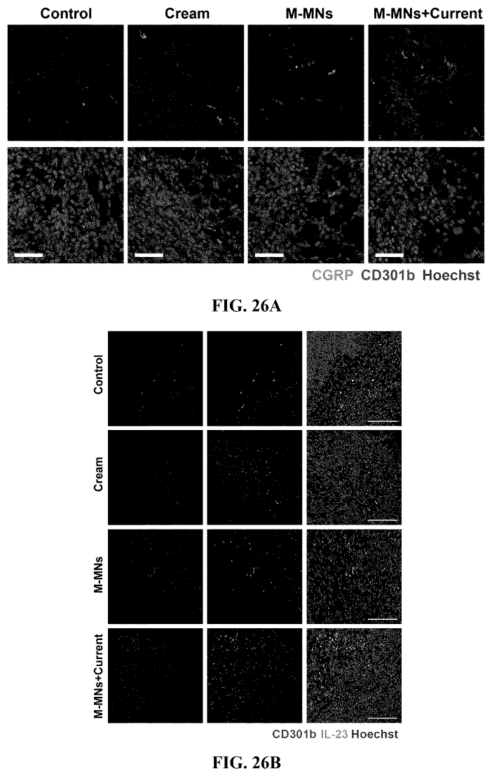

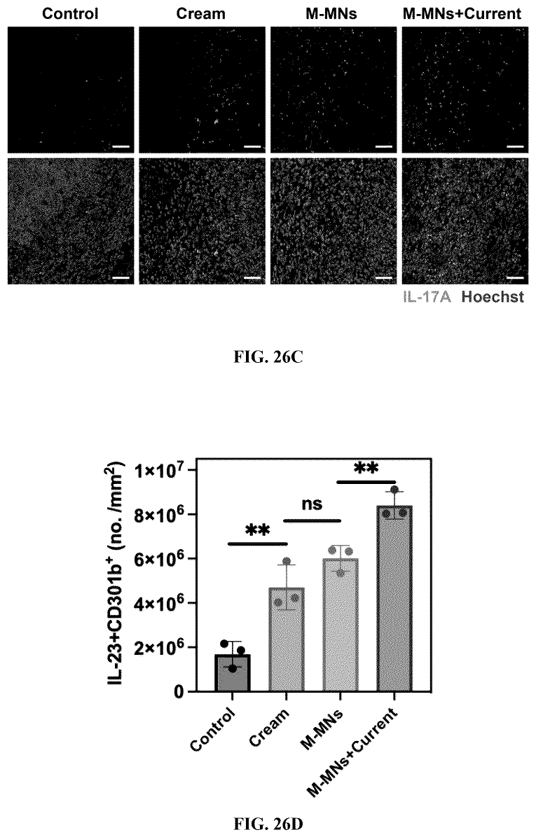

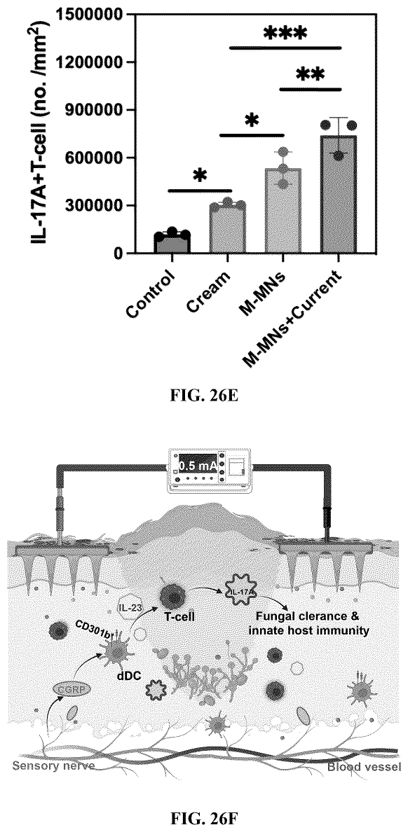

Furthermore, the present study also investigates whether CGRP can activate the innate immune response to induce the production of IL-23 and IL-17A, which in turn help clear fungal infections and elicit an immune-protective role. The immunofluorescence staining is performed to detect the expression of dDC, IL-23, and IL-17A. CD301b⁺ is a well-known marker for dDC cells found in the dermal layer of the skin. FIG. 26A illustrates the expression of CD301b⁺ DC cells and CGRP release under different treatment groups. The IHC images clearly show that the M-MNs+current treatment group expresses a significantly higher amount of CGRP and DC cells compared to the cream, M-MNs, and untreated control groups. The immunofluorescent staining consistently shows that it is the combination of electric current and the targeted delivery of miconazole via electrically-driven M-MNs, rather than miconazole cream or M-MNs alone, that triggers the release of CGRP from subcutaneous sensory nerves. This observation suggests that the higher concentration of miconazole nitrate induces a greater number of nociceptive neurons to produce CGRP, which in turn activates CD301b⁺ DC cells. The increased co-localization of CD301b⁺ DC cells with IL-23 in the MNs+current treatment group, as shown in FIG. 26B, confirms a higher upregulation of IL-23 compared to the other groups. It is also found that while miconazole cream and M-MNs alone increase the number of IL-23-expressing dDCs by approximately 2.5-fold, the combination of both results in a 4-fold increase in these cells (FIGS. 26B, 26D). As a result, the expression of IL-17A is found to be highest among all four tested groups (FIGS. 26C, 26E). Moreover, the enhanced production of IL-17A by γδ-T cells under the influence of MNs+current verifies the clearance of dead fungal tissue and the immune-protective role of the host.

Collectively, the data indicate that, in addition to the direct fungicidal effect on C. albicans, electrically-driven M-MNs also stimulate subcutaneous sensory nerves, utilizing the neuroimmune axis to eradicate microbial infection and promote protective immunity (FIG. 26F).

In conclusion, the present invention has successfully developed an electroconductive MN platform for the on-demand delivery of therapeutic agents to deep cutaneous tissues. It exhibits superior antimicrobial effects of the electrical-stimulated and miconazole-loaded MN patch compared to miconazole cream or drug-loaded MN patch alone. Moreover, the electroconductive MN platform reveals its ability to promote tissue healing and activates subcutaneous protective immunity through modulating the neuroimmune axis. Moreover, this innovative system offers several other advantages, including safety, affordability, and convenience, thereby alleviating the burden on healthcare system.

By combining the advantages of electricity and MN platforms, electroconductive MNs can deliver a high concentration of therapeutic agents (including but not limited to antimicrobial agents, small molecules, peptides, enzymes, extracellular vesicles, etc.) precisely to deep subcutaneous tissue layers within a short time frame (5 minutes). This contrasts with traditional MNs, which primarily follow a sustained release pattern. This unique capability of electroconductive MNs enhances their potential as an on-demand, non-invasive, self-administered transdermal drug delivery system.

In conclusion, the invention presents an electroconductive MN platform for targeted delivery of therapeutic agents to deep skin tissues. It demonstrated superior antimicrobial effects when using an electrical-stimulated, miconazole-loaded MN patch compared to traditional treatments. Additionally, the system promotes tissue healing and enhances subcutaneous immunity through the neuroimmune axis. This cost-effective, minimally invasive platform holds significant potential for clinical applications, offering benefits to millions of patients suffering from subcutaneous infections.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of 10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

The term “electroconductive microneedle” refers to a minimally invasive, needle-like structure fabricated from a polymer matrix embedded with conductive materials. These microneedles possess sufficient mechanical strength to penetrate the epidermal and dermal layers without deformation under applied force. When combined with an external electrical field, they facilitate the controlled and precise release of therapeutic agents into deep subcutaneous tissue layers.

The term “gelatin Methacryloyl (GelMA)” is a biofunctional, photo-crosslinkable biopolymer synthesized by chemically modifying gelatin with methacrylic anhydride. This polymer retains the natural biocompatibility and cell-interactive properties of gelatin while gaining the ability to form stable, crosslinked networks upon exposure to ultraviolet light.

The term “carbon nanotube” refers to cylindrical nanostructures composed of rolled graphene sheets with exceptional electrical conductivity, mechanical strength, and high surface area.

The term “electrical stimulation” refers to the application of a controlled electrical current to a biological system to induce therapeutic effects. In the invention, electrical stimulation is used to activate electroconductive microneedles, enabling on-demand drug release through mechanisms such as iontophoresis and electroporation. Parameters such as current density (e.g., 0.5 mA), voltage, and duration are precisely tuned to enhance drug permeation into deep tissues while avoiding pain or discomfort to the subject.

The term “subcutaneous” refers to the layer of tissue located beneath the dermis of the skin, primarily composed of fat and connective tissue. This layer serves as a protective cushion, insulates the body, and provides a route for nutrient and waste exchange.

The term “deep subcutaneous” refers to the innermost portion of the subcutaneous tissue, situated beneath the dermis and extending closer to the fascia covering muscles and other internal structures. In the context of the present invention, the term “deep subcutaneous area” specifically pertains to tissue located at a depth greater than 6 mm, typically ranging from 6 mm to 15 mm or more beneath the skin surface, depending on the anatomical location and individual variability. This region serves as the target site for delivering therapeutic agents to address conditions affecting deeper tissue layers, which are challenging to reach using conventional drug delivery methods.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### REFERENCES

The disclosures of the following references are incorporated by reference
ADDIN EN.REFLIST 1. J. Yang et al., Recent advances of microneedles used towards stimuli-responsive drug delivery, disease theranostics, and bioinspired applications. Chemical Engineering Journal 426, 130561 (2021).
2. B. J. Gowda et al., Stimuli-responsive microneedles as a transdermal drug delivery system: a demand-supply strategy. Biomacromolecules 23, 1519-1544 (2022).
3. US20170028184A1.
4. US20220193403A1.
5. WO2020060495A1.
6. US20210386985A1.
7. U.S. Pat. No. 9,737,247B2.
8. P. Basso et al., Deep tissue infection by an invasive human fungal pathogen requires lipid-based suppression of the IL-17 response. Cell Host & Microbe 30, 1589-1601. e1585 (2022).
9. S. Rawat, S. Vengurlekar, B. Rakesh, S. Jain, G. Srikarti, Transdermal delivery by iontophoresis. Indian journal of pharmaceutical sciences 70, 5 (2008).
10. R. Luo, J. Dai, J. Zhang, Z. Li, Accelerated skin wound healing by electrical stimulation. Advanced healthcare materials 10, 2100557 (2021).
11. J. Mi et al., Implantable electrical stimulation at dorsal root ganglions accelerates osteoporotic fracture healing via calcitonin gene-related peptide. Advanced Science 9, 2103005 (2022).

1. A polymer-based electroconductive microneedle patch for delivering a drug to a deep subcutaneous tissue, comprising:
a biocompatible substrate as a supporting structure;
a microneedle array comprises a plurality of polymer-based electroconductive microneedles affixed to the substrate, wherein the polymer comprises a photocrosslinkable biocompatible material reinforced with one or more electroconductive materials, the polymer and the photocrosslinkable biocompatible material have a ratio between 100:5 to 100:10; and
at least one therapeutic agent loaded onto the multiple polymer-based electroconductive microneedles,
wherein the microneedle array is configured to release the at least one therapeutic agent upon on-demand stimulation by an electrical current of 0.5 mA or above, so that the at least one therapeutic agent is able to be delivered to sites of deep cutaneous layers without causing any damage to surrounding tissues.

2. The polymer-based electroconductive microneedle patch of claim 1, wherein the photocrosslinkable biocompatible material comprises gelatin methacrylate (GelMA), hyaluronic acid methacrylate polymer, alginate methacrylate, or acrylates.

3. The polymer-based electroconductive microneedle patch of claim 1, wherein the one or more electroconductive materials comprise carbon nanotube, graphene, MXenes, graphene oxides, or reduced graphene oxides.

4. The polymer-based electroconductive microneedle patch of claim 3, wherein the one or more electroconductive materials provide enhanced electrical conductivity and mechanical reinforcement, achieving sufficient mechanical strength to penetrate the epidermis layer without deformation under a force of 0.5 N per needle.

5. The polymer-based electroconductive microneedle patch of claim 1, wherein the biocompatible substrate comprises biodegradable polymers selected from gelatin, alginates, collagen, dextran, chitosan, albumin, starch, cellulose, methyl cellulose, carboxymethyl cellulose, or hyaluronic acid.

6. The polymer-based electroconductive microneedle patch of claim 1, wherein the at least one therapeutic agent comprises antibiotics, peptides, nanoparticles, enzymes, extracellular vesicles, antifungal drugs, cationic therapeutic molecules, or a combination thereof.

7. The polymer-based electroconductive microneedle patch of claim 6, wherein the at least one therapeutic agent comprises miconazole nitrate, with a loading amount of approximately 400 μg per patch.

8. The polymer-based electroconductive microneedle patch of claim 1, wherein the plurality of polymer-based electroconductive microneedles has a height of 500-800 μm, a tip radius of 1-10 μm, and a base width of 200-400 μm.

9. The polymer-based electroconductive microneedle patch of claim 1, wherein the polymer-based electroconductive microneedle patch exhibits antimicrobial properties without causing local inflammation and accelerates wound healing by promoting collagen synthesis in fibroblasts.

10. The polymer-based electroconductive microneedle patch of claim 9, wherein the collagen synthesis in the fibroblasts is further enhanced by a sensory neuron-derived calcitonin gene-related peptide.

11. The polymer-based electroconductive microneedle patch of claim 1, wherein the polymer-based electroconductive microneedle patch releases at least 55% of the at least one therapeutic agent within 5 minutes under an applied current of 0.5 mA.

12. A method for manufacturing a polymer-based electroconductive microneedle patch, comprising:
synthesizing a gelatin methacrylate solution by reacting gelatin with methacrylic anhydride;
preparing a polymer matrix by dispersing carbon nanotubes into the gelatin methacrylate solution at a carbon nanotube concentration of 5.0 mg/mL to 10.0 mg/mL;
pouring the polymer matrix into a micromold;
photo-crosslinking the polymer matrix under UV light to form solidified microneedles; and
loading the solidified microneedles with at least one therapeutic agent.

13. The method of claim 12, wherein the at least one therapeutic agent comprises antibiotics, peptides, nanoparticles, enzymes, extracellular vesicles, antifungal drugs, cationic therapeutic molecules, or a combination thereof.

14. The method of claim 12, further comprising attaching the polymer-based electroconductive microneedle patch to a silver adhesive tape and connecting it to an external power source for electrical stimulation.

15. A method for treating deep subcutaneous diseases, conditions, and disorders, comprising:
administering the polymer-based electroconductive microneedle patch of claim 1 to a subject;
generating an electrical field by supplying a current density of 0.5 mA; and
releasing at least one therapeutic agent into a deep subcutaneous tissue in response to the electrical field,
wherein the polymer-based electroconductive microneedle patch is configured to release the at least one therapeutic agent upon on-demand stimulation, so that the at least one therapeutic agent is able to be delivered to sites of deep cutaneous layers without causing any damage to surrounding tissues, and
wherein the current applied to the polymer-based electroconductive microneedle patch contributes to transient loosening of tight junctions between the subject's skin cells, facilitating permeation of the at least one therapeutic agent into deeper skin layers.

16. The method of claim 15, wherein the deep subcutaneous diseases, conditions, and disorders comprise microbial infection, acne, cancer, diabetes, mycetoma, chromoblastomycosis, and keratitis.

17. The method of claim 16, wherein the deep subcutaneous diseases, conditions, and disorders is microbial infection, and the polymer-based electroconductive microneedle patch is configured to disrupt negatively charged microbial cell walls and polysaccharides matrix components of a biofilm, improving penetration of the at least one therapeutic agent into dense biofilm niches and eradicating the microbial infection.

18. The method of claim 15, wherein the polymer-based electroconductive microneedle patch provides an antimicrobial efficacy against fungal infections caused by Candida albicans, achieving a fungal clearance rate of at least 99.9%.

19. The method of claim 15, wherein the polymer-based electroconductive microneedle patch demonstrates biocompatibility with the subject's skin keratinocytes and sustain cell viability greater than 90% after administration.

20. The method of claim 15, wherein the electrical field activates nociceptive sensory neurons in the deep subcutaneous tissue, promoting immune responses via a neuroimmune axis.
